(19) United States
(12) Patent Application Publication
(10) Pub. No.: US 2012/0324602 A1
(43) Pub. Date: Dec. 20, 2012

(54) COMPLEXES OF AN3-INTERACTING PROTEINS AND THEIR USE FOR PLANT GROWTH PROMOTION

(75) Inventors: Geert De Jaeger, Evergem (BE); Dirk Inzé, Moorsel-Aalst (BE); Aurine Verkest, Gent (BE)

(73) Assignee: BASF PLANT SCIENCE COMPANY GMBH, LUDWIGSHAFEN (DE)

(21) Appl. No.: 13/579,708

(22) PCT Filed: Feb. 21, 2011
(86) PCT No.: PCT/EP11/52525

§ 371 Date: Aug. 30, 2012

(30) Foreign Application Priority Data
Feb. 22, 2010 (EP) 10154219.9

(51) Int. Cl. C07K 19/00; A01H 1/06; A01P 21/00; A01N 37/46

(52) U.S. Cl. 800/290; 530/370; 504/335

(57) ABSTRACT

The present invention relates to protein complexes based on AN3-interactors, more specifically interactors that are plant variants subunits of the SWI/SNF complex, and proteins that interact with those subunits, preferably in an AN3 free protein complex. It relates further to the use of the complexes to promote plant growth, and to a method for stimulating the complex formation, by overexpressing at least one, preferably at least two members of a complex.

The present invention relates to protein complexes based on AN3-interactors, more specifically interactors that are plant variants subunits of the SWI/SNF complex, and proteins that interact with those subunits, preferably in an AN3 free protein complex. It relates further to the use of the complexes to promote plant growth, and to a method for stimulating the complex formation, by overexpressing at least one, preferably at least two members of a complex.

The demand for more plant derived products has spectacularly increased. In the near future the challenge for agriculture will be to fulfill the growing demands for feed and food in a sustainable manner. Moreover plants start to play an important role as energy sources. To cope with these major challenges, a profound increase in plant yield will have to be achieved. Biomass production is a multi-factorial system in which a plethora of processes are fed into the activity of meristems that give rise to new cells, tissues, and organs. Although a considerable amount of research on yield performance is being performed little is known about the molecular networks underpinning yield (Van Camp, 2005). Many genes have been described in Arabidopsis thaliana that, when mutated or ectopically expressed, result in the formation of larger structures, such as leaves or roots. These so-called “intrinsic yield genes” are involved in many different processes whose interrelationship is mostly unknown.

One of these “intrinsic yield genes”, AN3 (also known as GIF1), was identified in search of GRF (growth regulating factor) interactors (Kim and Kende, 2004) and by analysis of narrow-leaf Arabidopsis mutants (Horiguchi et al., 2005). AN3 is a homolog of the human SYT (synovial sarcoma translocation) protein and is encoded by a small gene family in the Arabidopsis genome. SYT is a transcription co-activator whose biological function, despite the implication of its chromosomal translocation in tumorigenesis, is still unclear (Clark et al., 1994; de Bruijn et al., 1996). Using the yeast GAL4 system, AN3 was shown to possess transactivation activity (Kim and Kende, 2004). This together with yeast two-hybrid and in vitro binding assays demonstrating interaction of AN3 with several GRFs (Kim and Kende, 2004; Horiguchi et al., 2005), suggests a role of AN3 as transcription co-activator of GRFs. GRF (growth regulating factor) genes occur in the genomes of all seed plants thus far examined and encode putative transcription factors that play a regulatory role in growth and development of leaves (Kim et al., 2003). In support of a GRF and AN3 transcription activator and co-activator complex, grf and an3 mutants display similar phenotypes, and combinations of grf and an3 mutations showed a cooperative effect (Kim and Kende, 2004). The an3 mutant narrow-leaf phenotype is shown to result of a reduction in cell numbers. Moreover, ectopic expression of AN3 resulted in transgenic plants with larger leaves consisting of more cells, indicating that AN3 controls both cell number and organ size (Horiguchi et al., 2005). Although the function of AN3 in plant growth regulation is not known, these results show that AN3 fulfills the requirements of an “intrinsic yield gene”.

al., 2006) but so far none of the identified genes have been associated with stimulation of plant growth.

In our ambition to decipher the molecular network underpinning yield enhancement mechanism a genome-wide protein centered approach was undertaken to study AN3 interacting proteins in Arabidopsis thaliana cell suspension cultures. The tandem affinity purification (TAP) technology combined with mass spectrometry (MS) based protein identification resulted in the isolation and identification of 25 AN3 interacting proteins that may function in the regulation of plant growth (Table 1). We isolated several proteins belonging to multiprotein complexes. Moreover, many interactors are completely uncharacterized. Reports on few of the AN3 interactors show that they are implicated in several developmental processes (Wagner & Meyerowitz, 2002; Meagher et al., 2005; Sarnowski et al., 2005; Hurtado et al., 2006; Kwon et al., 2006) but so far none of the identified genes have been associated with stimulation of plant growth.

TABLE 1
Interactors of AN3 identified by TAP analysis on
cell suspension cultures.
Table 1. 35S-AN3 (8 experiments)

| | | TAP | | |
|---|---|---|---|---|
| AT number | Protein name | total | C-GS | N-GS |
| AT5G28640 | AN3 | 4 | 3 | 1 |
| AT4G16143 | importin alpha-2 (IMPA2) | 5 | 4 | 1 |
| AT3G06720 | importin alpha-1 subunit (IMPA1) | 4 | 4 | / |
| AT5G53480 | importin beta-2 | 4 | 4 | / |
| AT1G09270 | importin alpha-1 subunit (IMPA4) | 1 | 1 | / |
| AT2G28290 | chromatin remodeling protein | 8 | 4 | 4 |
| | (SYD) | | | |
| AT3G60830 | actin-related protein 7 (ARP7) | 7 | 4 | 3 |
| AT2G46020 | SNF2 protein (BRM) | 6 | 4 | 2 |
| AT1G18450 | actin-related protein 4 (ARP4) | 4 | 4 | / |
| AT1G21700 | SWIRM domain-containing protein | 4 | 4 | / |
| | (SWI3C) | | | |
| AT5G14170 | SWIB complex BAF60b domain- | 4 | 4 | / |
| | containing protein (Swp73B) | | | |
| AT4G17330 | G2484-1, agenet domain- | 6 | 4 | 2 |
| | containing protein | | | |
| AT3G15000 | expressed protein, similar to DAG | 6 | 3 | 3 |
| | protein | | | |
| AT5G55210 | expressed protein | 4 | 4 | / |
| AT5G17510 | expressed protein | 3 | 3 | / |
| AT2G16570 | ATASE (Gln Phosphoribosyl | 2 | / | 2 |
| | Pyrophospate Amidotransferase 1) | | | |
| AT4G35550 | homeobox-leucine zipper protein | 1 | 1 | / |
| | (HB-2)/HD-ZIP protein | | | |
| AT1G20670 | DNA-binding bromodomain- | 1 | 1 | / |
| | containing protein | | | |
| AT3G55220 | splicing factor | 1 | 1 | / |
| AT2G46340 | phytochrome A supressor spa1 | 1 | 1 | / |
| | (SPA1) | | | |
| AT5G13030 | expressed protein | 1 | 1 | / |
| AT5G17330 | GAD (Glutamate decarboxylase); | 1 | / | 1 |
| | calmodulin binding | | | |
| AT1G80480 | PTAC17 (PLASTID | 1 | / | 1 |
| | TRANSCRIPTIONALLY | | | |
| | ACTIVE17) | | | |
| AT1G43800 | acyl-(acyl-carrier-protein) | 1 | / | 1 |
| | desaturase | | | |
| AT5G45620 | 26S proteasome regulatory subunit | 1 | 1 | / |
| | (RPN9) | | | |

TAP total gives the total number of time that an interactor was co-purified;
C-GS and N-GS refers to whether a C or N terminal GS-tag was used in the experiment.

Several of the AN3p interactors were homologues of subunits of the SWI/SNF type chromatin remodeling complex (Thaete et al., 1999; Ishida et al., 2004). Recently, it was shown in mammalian cells that the SWI/SNF ATP-dependent chromatin remodeling complex plays an important role in cell differentiation and proliferation in mammalian cells (Riesman et al., 2009) Surprisingly we found that plant variants of subunits of the SWI/SNF complex, and their interactors play an important role in plant growth, and can be used to increase plant yield. A first aspect of the invention is an isolated protein complex, preferably an AN3p-free protein complex, comprising at least a plant variant of a SWI/SNF3 subunit, said subunit capable of interacting with AN3p, and one of more proteins interacting with said variant SWI/SNF3 subunit.

An AN3p-free protein complex, as used here, means that AN3p is not present in the complex as isolated; however, one or more subunits of the complex may be capable of interacting with AN3, and AN3 may be capable of interacting with the complex as a whole. In a preferred embodiment, the complex according to the invention is not longer capable of interacting with AN3, whereby the protein interacting with the plant variant of the SWI/SNF3 subunit directly or indirectly inhibits binding of AN3p to said variant. Direct inhibition of AN3p binding may be caused by, as a non-limiting example, by binding to the same domain; indirect inhibition of AN3p binding may be caused, as a non-limiting example, by conformational changes in said variant upon binding with its interactor. Plant variants of SWI/SNF chromatin remodeling complex subunits are known to the person skilled in the art, and have been described, amongst others, by Jerzmanowski (2007), hereby incorporated by reference. Variants, as used here, are including, but not limited to homologues, orthologues and paralogues of said cell cycle related proteins. “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene. Preferably, said homologue, orthologue or paralogue has a sequence identity at protein level of at least 30%, preferably at least 40%, preferably 50%, 51%, 52%, 53%, 54% or 55%, 56%, 57%, 58%, 59%, preferably 60%, 61%, 62%, 63%, 64%, 65%, 66%, 67%, 68%, 69%, more preferably 70%, 71%, 72%, 73%, 74%, 75%, 76%, 77%, 78%, 79%, even more preferably 80%, 81%, 82%, 83%, 84%, 85%, 86%, 87%, 88%, 89% most preferably 90%, 91%, 92%, 93%, 94%, 95%, 96%, 97%, 98%, 99% or more as measured in a BLASTp (Altschul et al., 1997; Altschul et al., 2005). A plant as used here can be any plant. In one preferred embodiment, said plant is Arabidopsis thaliana. In another preferred embodiment, said plant is a crop plant, preferably a monocot or a cereal, even more preferably it is a cereal selected from the group consisting of rice, maize, wheat, barley, millet, rye, sorghum and oats.

Preferably, said plant variant of the SWI/SNF chromatin remodelling complex is selected from the group consisting of proteins encoded by AT1G18450 (ARP4), AT3G60830 (ARP7), AT5G14170 (Swp73B) and AT1G21700 (SWI3C), or a variant thereof.

On preferred embodiment is an isolated protein complex, preferably an isolated AN3-protein free protein complex, comprising at least ARP4p or a variant thereof and a protein selected from the group encoded by AT5G45600, AT1G76380, AT3G01890, AT5G26360, AT5G14240, AT1G47128, AT2G27100, AT5G55040, AT3G03460 and AT1G54390, or a variant thereof. Another preferred embodiment is an isolated protein complex, preferably an isolated AN3-protein free protein complex, comprising at least ARP7p or a variant thereof and a protein selected from the group encoded by AT3G20050, AT5G14240, AT4G22320, AT5G26360, AT3G02530, AT3G18190, AT3G03960, AT3G08580, AT4G14880 and AT1G07820, or a variant thereof.

Another preferred embodiment is an isolated protein complex, preferably an isolated AN3-protein free protein complex, comprising at least Swp73 Bp or a variant thereof and a protein selected from the group encoded by AT2G47620, AT2G33610, AT3G17590, AT4G34430, AT1G32730, AT3G22990, AT1G06500, AT1G47128, AT3G18380, AT3G06010, AT1G58025, AT5G03290, AT5G55040, AT3G50000, AT4G28520, AT5G44120 and AT4G22320, or a variant thereof.

Still another preferred embodiment is an isolated protein complex, preferably an isolated AN3-protein free protein complex, comprising at least SWI3Cp and a protein selected from the group encoded by AT3G01890, AT1G76380, AT3G03460, AT4G22320, AT1G11840, AT4G14880 and AT4G04740, or a variant thereof.

Another aspect of the invention is the use of a protein complex according to the invention to modulate plant growth and/or plant yield. Preferably, said modulation is an increase of plant growth and/or yield. Preferably, increase of growth is measured as an increase of biomass production. “Yield” refers to a situation where only a part of the plant, preferably an economical important part of the plant, such as the leaves, roots or seeds, is increased in biomass. The term “increase” as used here means least a 5%, 6%, 7%, 8%, 9% or 10%, preferably at least 15% or 20%, more preferably 25%, 30%, 35% or 40% more yield and/or growth in comparison to control plants as defined herein. Increase of plant growth, as used here, is preferably measured as increase of any one or more of total plant biomass, leaf biomass, root biomass and seed biomass. In one preferred embodiment, said increase is an increase in total plant biomass. In a preferred embodiment, said plant is a crop plant, preferably a monocot or a cereal, even more preferably it is a cereal selected from the group consisting of rice, maize, wheat, barley, millet, rye, sorghum and oats.

Still another aspect of the invention is a method to promote the formation of a protein complex according to the inventions, comprising the overexpression of at least one protein, preferably at least two proteins of said complex. Overexpression of a target gene can be obtained by transfer of a genetic construct, intended for said overexpression into a plant. The transfer of foreign genes into the genome of a plant is called transformation. Transformation of plant species is a fairly routine technique known to the person skilled in the art. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include, but are not limited to agrobacterium mediated transformation, the use of liposomes, electroporation, chemicals that increase free DNA uptake, injection of the DNA directly into the plant, particle gun bombardment, transformation using viruses or pollen and microprojection. Preferably, said overexpression results in an increase of plant growth and/or yield. Increase of plant growth and/or yield is measured by comparing the test plant, comprising a gene used according to the invention, with the parental, non-transformed plant, grown under the same conditions as control.

Still another aspect of the invention is a method to inhibit the formation of a protein complex according to the inventions, comprising the repression of the expression of at least one protein, preferably at least two proteins of said complex. Inhibition of complex formation can be desirable in cases where the complex exerts a growth limiting effect. Repression of expression of a target gene can be obtained by transfer of a genetic construct, intended for said repression of expression into a plant. Methods for repressing the expression in plants are known to the person skilled in the art and include, but are not limited to the use of RNAi, anti-sense RNA and gene silencing.

### BRIEF DESCRIPTION OF THE FIGURES

FIG. 1: leaf phenotype of 2 SWIRM overexpressing lines. A) total rosette area. B) area of individual leaves. Plants were grown in vitro for 21 days. SWIRM is an alternative name for SWI3C.

### EXAMPLES

### Materials and Methods to the Examples

### Vector Construction for AN3 Interactors

Construction of N- and C-terminal GS-tagged GFP and AN3 under the control of the 35S (CaMV) promoter was obtained by Multisite Gateway LR reactions. The coding regions, without (−) and with (+) stopcodon, were amplified by polymerase chain reaction (PCR) and cloned into the Gateway pDONR221 vector (Invitrogen) resulting in pEntryL1L2-GFP(−), pEntryL1L2-GFP(+), pEntryL1L2-AN3(−) and pEntryL1L2-AN3(+). The Pro_{35S}:GFP-GS- and Pro_{3S}:AN3-GS-containing plant transformation vectors were obtained by Multisite Gateway LR reaction between pEntryL4R1-Pro₃₅ₛ, pEntryL1L2-GFP(−) or pEntryL1L2-AN3(−), and pEntryR2L3-GS and the destination vector pKCTAP, respectively (Van Leene et al., 2007). To obtain the Pro35S:GS-GFP and Pro35S:GS-AN3 vectors Multisite LR recombination between pEntryL4L3-Pro_{35S} and pEntryL1L2-GFP(+) or pEntryL1L2-AN3(+) with pKNGSTAP occurred. All entry and destination vectors were checked by sequence analysis. Expression vectors were transformed to Agrobacterium tumefaciens strain C58C1Rif^{R} (pMP90) by electroporation. Transformed bacteria were selected on yeast extract broth plates containing 100 μg/mL rifampicin, 40 μg/mL gentamicin, and 100 μg/mL spectinomycin.

### Vector Construction for ARP4, ARP7, Swp73B and SWI3C Interactors

Construction of N- and C-terminal GS-tagged ARP4, ARP7, Swp73B and SWI3C under the control of the 35S (CaMV) promoter was obtained by Multisite Gateway LR reactions. The coding regions, without (−) and with (+) stopcodon, were amplified by polymerase chain reaction (PCR) and cloned into the Gateway pDONR221 vector (Invitrogen) resulting in pEntryL1L2-ARP4(−), pEntryL1L2-ARP4(+), pEntryL1L2-ARP7(−), pEntryL1L2-ARP7(+), pEntryL1L2-Swp73B(−), pEntryL1L2-Swp73B(+), pEntryL1L2-SWI3C(−) and pEntryL1L2-SWI3C(+). The Pro_{35S}:ARP4-GS-, Pro_{35S}:ARP7-GS-, Pro_{35S}:Swp73B-GS- and Pro_{35S}:SWI3C-GS-containing plant transformation vectors were obtained by Multisite Gateway LR reaction between pEntryL4R1—Pro_{35S}, pEntryL1L2-ARP4(−), pEntryL1L2-ARP7(−), pEntryL1L2-Swp73B(−) or pEntryL1L2-SWI3C(−), and pEntryR2L3-GS and the destination vector pKCTAP, respectively (Van Leene et al., 2007). To obtain the Pro35S:GS-ARP4, Pro35S:GS-ARP7, Pro35S:GS-Swp73B and Pro35S:GS-SWI3C vectors Multisite LR recombination between pEntryL4L3-Pro_{35S} and pEntryL1L2-ARP4(+), pEntryL1L2-ARP7(+), pEntryL1L2-Swp73B(+) or pEntryL1L2-SWI3C(+) with pKNGSTAP occurred.

All entry and destination vectors were checked by sequence analysis. Expression vectors were transformed to Agrobacterium tumefaciens strain C58C1Rif^{R} (pMP90) by electroporation. Transformed bacteria were selected on yeast extract broth plates containing 100 μg/mL rifampicin, 40 μg/mL gentamicin, and 100 μg/mL spectinomycin.

### Cell Suspension Cultivation

Wild-type and transgenic Arabidopsis thaliana cell suspension PSB-D cultures were maintained in 50 mL MSMO medium (4.43 g/L MSMO, Sigma-Aldrich), 30 g/L sucrose, 0.5 mg/L NAA, 0.05 mg/L kinetin, pH 5.7 adjusted with 1M KOH) at 25° C. in the dark, by gentle agitation (130 rpm). Every 7 days the cells were subcultured in fresh medium at a 1/10 dilution.

### Cell Culture Transformation

The Arabidopsis culture was transformed by Agrobacterium co-cultivation as described previously (Van Leene et al., 2007). The Agrobacterium culture exponentially growing in YEB (OD₆₀₀ between 1.0 and 1.5) was washed three times by centrifugation (10 min at 5000 rpm) with an equal volume MSMO medium and resuspended in cell suspension growing medium until an OD₆₀₀ of 1.0. Two days after subcultivation, 3 mL suspension culture was incubated with 200 μL washed Agrobacteria and 200 μM acetoseringone, for 48 h in the dark at 25° C. with gentle agitation (130 rpm). Two days after co-cultivation, 7 mL MSMO containing a mix of three antibiotics (25 μg/mL kanamycin, 500 μg/mL carbenicellin, and 500 μg/mL vancomycin) was added to the cell cultures and grown further in suspension under standard conditions (25° C., 130 rpm and continuous darkness). The stable transgenic cultures were selected by sequentional dilution in a 1:5 and 1:10 ratio in 50 mL fresh MSMO medium containing the antibiotics mix, respectively at 11, and 18 days post co-cultivation. After counter selecting the bacteria, the transgenic plant cells were further subcultured weekly in a 1:5 ratio in 50 mL MSMO medium containing 25 μg/mL kanamycin for two more weeks. Thereafter the cells were weekly subcultured in fresh medium at a 1/10 dilution.

### Expression Analysis of Cell Suspension Cultures

Transgene expression was analyzed in a total protein extract derived from exponentially growing cells, harvested two days after subculturing. Equal amounts of total protein were separated on 12% SDS-PAGE gels and blotted onto Immobilon-P membranes (Millipore, Bedford, Mass.). Protein gel blots were blocked in 3% skim milk in 20 mM Tris-HCl, pH 7.4, 150 mM NaCl, and 0.1% Triton X-100. For detection of GS-tagged proteins, blots were incubated with human blood plasma followed by incubation with anti-human IgG coupled to horseradish peroxidase (HRP; GE-Healthcare). Protein gel blots were developed by Chemiluminiscent detection (Perkin Elmer, Norwalk, Conn.).

### Protein Extract Preparation

Cell material (15 g) was grinded to homogeneity in liquid nitrogen. Crude protein extract were prepared in an equal volume (w/v) of extraction buffer (25 mM Tris-HCl, pH 7.6, 15 mM MgCl₂, 5 mM EGTA, 150 mM NaCl, 15 mM p-nitrophenylphosphate, 60 mM β-glycerophosphate, 0.1% (v/v) Nonidet P-40 (NP-40), 0.1 mM sodium vanadate, 1 mM NaF, 1 mM DTT, 1 mM PMSF, 10 μg/mL leupeptin, 10 μg/mL aprotinin, 5 μg/mL antipain, 5 μg/mL chymostatin, 5 μg/mL pepstatin, 10 μg/mL soybean trypsin inhibitor, 0.1 mM benzamidine, 1 μM trans-epoxysuccinyl-L-leucylamido-(4-guanidino)butane (E64), 5% (v/v) ethylene glycol) using an Ultra-Turrax T25 mixer (IKA Works, Wilmington, N.C.) at 4° C. The soluble protein fraction was obtained by a two-step centrifugation at 36900 g for 20 min and at 178000 g for 45 min, at 4° C. The extract was passed through a 0.45 μm filter (Alltech, Deerfield, Ill.) and the protein content was determined with the Protein Assay kit (Bio-Rad, Hercules, Calif.).

### Tandem Affinity Purification

Purifications were performed as described by Bürckstümmer et al. (2006), with some modifications. Briefly, 200 mg total protein extract was incubated for 1 h at 4° C. under gentle rotation with 100 μL IgG Sepharose 6 Fast Flow Flow beads (GE-Healthcare, Little Chalfont, UK), pre-equilibrated with 3 mL extraction buffer. The IgG Sepharose beads were transferred to a 1 mL Mobicol column (MoBiTec, Goettingen, Germany) and washed with 10 mL IgG wash buffer (10 mM Tris-HCl, pH 8.0, 150 mM NaCl, 0.1% NP-40, 5% ethylene glycol) and 5 mL Tobacco (Nicotiana tabacum L.) Etch Virus (TEV) buffer (10 mM Tris-HCl, pH 8.0, 150 mM NaCl, 0.1% (v/v) NP-40, 0.5 mM EDTA, 1 mM PMSF, 1 μM E64, 5% (v/v) ethylene glycol). Bound complexes were eluted via AcTEV digest (2×100 U, Invitrogen) for 1 h at 16° C. The IgG eluted fraction was incubated for 1 h at 4° C. under gentle rotation with 100 μL Streptavidin resin (Stratagene, La Jolla, Calif.), pre-equilibrated with 3 mL TEV buffer. The Streptavidin beads were packed in a Mobicol column, and washed with 10 mL TEV buffer. Bound complexes were eluted with 1 mL streptavidin elution buffer (10 mM Tris-HCl, pH 8.0, 150 mM NaCl, 0.1% (v/v) NP-40, 0.5 mM EDTA, 1 mM PMSF, 1 μM E64, 5% (v/v) ethylene glycol, 20 mM Desthiobiotin), and precipitated using TCA (25% v/v). The protein pellet was washed twice with ice-cold aceton containing 50 mM HCl, redissolved in sample buffer and separated on 4-12% gradient NuPAGE gels (Invitrogen). Proteins were visualized with colloidal Coomassie brilliant blue staining.

### Proteolysis and Peptide Isolation

After destaining, gel slabs were washed for 1 hour in H₂O, polypeptide disulfide bridges were reduced for 40 min in 25 mL of 6.66 mM DTT in 50 mM NH₄HCO₃ and sequentially the thiol groups were alkylated for 30 min in 25 mL 55 mM IAM in 50 mM NH₄HCO₃. After washing the gel slabs 3 times with water, complete lanes from the protein gels were cut into slices, collected in microtiter plates and treated essentially as described before with minor modifications (Van Leene et al., 2007). Per microtiterplate well, dehydrated gel particles were rehydrated in 20 μL digest buffer containing 250 ng trypsin (MS Gold; Promega, Madison, Wis.), 50 mM NH₄HCO₃ and 10% CH₃CN (v/v) for 30 min at 4° C. After adding 10 μL of a buffer containing 50 mM NH₄HCO₃ and 10% CH₃CN (v/v), proteins were digested at 37° C. for 3 hours. The resulting peptides were concentrated and desalted with microcolumn solid phase tips (PerfectPure™ C18 tip, 200 mL bed volume; Eppendorf, Hamburg, Germany) and eluted directly onto a MALDI target plate (Opti-TOF™384 Well Insert; Applied Biosystems, Foster City, Calif.) using 1.2 μL of 50% CH₃CN: 0.1% CF₃COOH solution saturated with α-cyano-4-hydroxycinnamic acid and spiked with 20 fmole/μL Glu1-Fibrinopeptide B (Sigma-Aldrich), 20 fmole/μL des-Pro2-Bradykinin (Sigma-Aldrich), and 20 fmole/μL Adrenocorticotropic Hormone Fragment 18-39 human (Sigma-Aldrich).

### Acquisition of Mass Spectra

A MALDI-tandem MS instrument (4800 Proteomics Analyzer; Applied Biosystems) was used to acquire peptide mass fingerprints and subsequent 1 kV CID fragmentation spectra of selected peptides. Peptide mass spectra and peptide sequence spectra were obtained using the settings essentially as presented in Van Leene et al. (2007). Each MALDI plate was calibrated according to the manufacturers' specifications. All peptide mass fingerprinting (PMF) spectra were internally calibrated with three internal standards at m/z 963.516 (des-Pro2-Bradykinin), m/z 1570.677 (Glu1-Fibrinopeptide B), and m/z 2465, 198 (Adrenocorticotropic Hormone Fragment 18-39) resulting in an average mass accuracy of 5 ppm±10 ppm for each analyzed peptide spot on the analyzed MALDI targets. Using the individual PMF spectra, up to sixteen peptides, exceeding a signal-to-noise ratio of 20 that passed through a mass exclusion filter were submitted to fragmentation analysis.

### MS-Based Protein Homology Identification

PMF spectra and the peptide sequence spectra of each sample were processed using the accompanied software suite (GPS Explorer 3.6, Applied Biosystems) with parameter settings essentially as described in Van Leene et al. (2007). Data search files were generated and submitted for protein homology identification by using a local database search engine (Mascot 2.1, Matrix Science). An in-house nonredundant Arabidopsis protein database called SNAPS Arabidopsis thaliana version 0.4 (SNAPS=Simple Nonredundant Assembly of Protein Sequences, 77488 sequence entries, 30468560 residues; available at http://www.ptools.ua.ac.be/snaps) was compiled from nine public databases. Protein homology identifications of the top hit (first rank) with a relative score exceeding 95% probability were retained. Additional positive identifications (second rank and more) were retained when the score exceeded the 98% probability threshold.

### Example 1

### Identification of AN3 Interactors

In order to identify the interaction partners of AN3 in vivo, we performed tandem affinity (TAP) purifications on N- and C-terminal GS-fusions of AN3 ectopically expressed under control of the constitutive 35SCaMV promoter in transgenic Arabidopsis suspension cultures. Two independent TAP purifications were performed on extracts from AN3-GS and GS-AN3 lines, harvested two days after sub-culturing into fresh medium. The affinity purified proteins were separated on a 4-12% NuPAGE gel and stained with Coomassie Brilliant Blue. Protein bands were cut, in-gel digested with trypsin and subjected to MALDI-TOF/TOF mass spectrometry for protein identification. After subtracting background proteins, identified by the control purifications (Van Leene et al., 2007), from the obtained hit list we identified 25 AN3 interacting proteins, other than AN3 itself (Table 1). 9 proteins were identified only in one out of 8 TAP experiments.

### Example 2

### Identification of ARP4 Interactors

ARP4 interactors were identified according to the methods described above. The results are summarized in Table 2. Apart from proteins, already identified in the AN3 complex (Table 1), several novel interactors were identified.

TABLE 2
Interactors of ARP4, identified by TAP analysis on
cell suspension cultures.
Table 2. 35S-ARP4 (4 experiments)

| | | TAP | | |
|---|---|---|---|---|
| AT number | Protein name | total | C-GS | N-GS |
| AT1G18450 | ARP4 | 4 | 2 | 2 |
| AT3G60830 | ARP7 | 4 | 2 | 2 |
| AT5G45600 | TAF14B, GAS41 | 2 | / | 2 |
| AT5G14170 | Swp73B | 2 | 2 | / |
| AT1G21700 | SWI3C | 2 | 2 | / |
| AT1G20670 | DNA-binding bromodomain- | 2 | 2 | / |
| | containing protein | | | |
| AT1G76380 | DNA-binding bromodomain- | 2 | 2 | / |
| | containing protein | | | |
| AT2G46020 | BRM | 2 | 2 | / |
| AT3G01890 | Swp73A | 2 | 2 | / |
| AT5G26360 | chaperonin | 2 | / | 2 |
| AT5G14240 | protein coding | 2 | / | 2 |
| AT5G17510 | expressed protein | 2 | 2 | / |
| AT1G47128 | RD21 (RESPONSIVE TO | 2 | 2 | / |
| | DEHYDRATION 21) | | | |
| AT2G27100 | SE (SERRATE) | 2 | 1 | 1 |
| AT5G55040 | DNA-binding bromodomain- | 1 | 1 | / |
| | containing protein | | | |
| AT5G55210 | expressed protein, similar to | 1 | 1 | / |
| | At4g22320 | | | |
| AT3G03460 | unknown protein | 1 | 1 | / |
| AT1G54390 | PHD finger protein | 1 | / | 1 |

TAP total gives the total number of time that an interactor was co-purified;
C-GS and N-GS refers to whether a C or N terminal GS-tag was used in the experiment.

### Example 3

### Identification of ARP7 Interactors

ARP7 interactors were identified according to the methods described above. The results are summarized in Table 3. ARP4 and At5g55210 were also identified as AN3 interactors (Table 1). It is interesting to note that the ARP4-ARP7 interaction is also identified using the ARP4 screening, confirming the reliability of the Tap-tag method. At5g55210 was also identified as AN3 as well as ARP4 interactor (Table 1 & 2).

TABLE 3
Interactors of ARP7, identified by TAP analysis on
cell suspension cultures.
Table 3. 35S-ARP7 (4 experiments)

| | | TAP | | |
|---|---|---|---|---|
| AT number | Protein name | total | C-GS | N-GS |
| AT3G60830 | ARP7 | 4 | 2 | 2 |
| AT3G20050 | TCP-1 (Arabidopsis thaliana T- | 2 | / | 2 |
| | complex protein 1 alpha subunit) | | | |
| AT5G14240 | protein coding | 2 | / | 2 |
| AT5G55210 | expressed protein, similar to | 2 | 2 | / |
| | At4g22320 | | | |
| AT4G22320 | unknown, similar to At5g55210 | 4 | 2 | 2 |
| AT5G26360 | chaperonin | 2 | / | 2 |
| AT3G02530 | chaperonin | 2 | / | 2 |
| AT3G18190 | chaperonin, similar to At3g03960 | 2 | / | 2 |
| AT3G03960 | chaperonin, similar to At3g18190 | 2 | / | 2 |
| AT1G18450 | ARP4 | 1 | / | 1 |
| AT3G08580 | AAC1 (ADP/ATP CARRIER 1) | 1 | 1 | / |
| AT4G14880 | OASA1 (O-ACETYLSERINE | 1 | 1 | / |
| | (THIOL) LYASE (OAS-TL) | | | |
| | ISOFORM A1) | | | |
| AT1G07820 | HIS4 | 1 | 1 | / |

TAP total gives the total number of time that an interactor was co-purified;
C-GS and N-GS refers to whether a C or N terminal GS-tag was used in the experiment.

### Example 4

### Identification of Swp73B Interactors

Swp73B interactors were identified according to the methods described above. The results are summarized in Table 4. Except for SYD, all AN3 interacting proteins of the SWI/SNF complex are interacting with Swp73B. Apart from those proteins, most of the other proteins show only interaction with Swp73B and not with the other proteins of the SWI/SNF complex used in the tap tag experiments (ARP4, ARP7 and SWI3C)

TABLE 4
Interactors of Swp73B, identified by TAP analysis on
cell suspension cultures.
Table 4. 35S-Swp73B (5 experiments)

| | | TAP | | |
|---|---|---|---|---|
| AT number | Protein name | total | C-GS | N-GS |
| AT5G14170 | Swp738 | 5 | 2 | 3 |
| AT2G47620 | SWI3A | 5 | 2 | 3 |
| AT1G20670 | DNA-binding bromodomain- | 5 | 2 | 3 |
| | containing protein, similar to | | | |
| | At5g55040 | | | |
| AT2G33610 | SWI3B | 4 | 2 | 2 |
| AT3G60830 | ARP7 | 4 | 2 | 2 |
| AT3G17590 | BSH | 4 | 2 | 2 |
| AT1G21700 | SWI3C | 4 | 1 | 3 |
| AT4G34430 | SWI3D | 4 | 1 | 3 |
| AT1G32730 | electron carrier | 4 | 2 | 2 |
| AT5G17510 | expressed protein | 4 | 2 | 2 |
| AT3G22990 | LFR (armadillo-repeat protein) | 4 | 2 | 2 |
| AT5G55210 | expressed protein, similar to | 4 | 2 | 2 |
| | At4g22320 | | | |
| AT2G46020 | BRM | 3 | 1 | 2 |
| AT1G18450 | ARP4 | 3 | 1 | 2 |
| AT1G06500 | unknown protein | 3 | 1 | 2 |
| AT1G47128 | RD21 (RESPONSIVE TO | 4 | 2 | 2 |
| | DEHYDRATION 21) | | | |
| AT3G18380 | transcription factor | 2 | / | 2 |
| AT3G06010 | CHR12 | 1 | / | 1 |
| AT1G58025 | DNA-binding bromodomain- | 1 | 1 | / |
| | containing protein | | | |
| AT5G03290 | isocitrate dehydrogenase | 1 | 1 | / |
| AT5G55040 | DNA-binding bromodomain- | 1 | 1 | 1 |
| | containing protein, similar to | | | |
| | At1g20670 | | | |
| AT3G50000 | CKA2 (casein kinase II alpha chain | 1 | / | 1 |
| | 2) | | | |
| AT4G28520 | CRU3 (CRUCIFERIN 3) | 1 | / | 1 |
| AT5G44120 | CRU1 (CRUCIFERINA) | 1 | / | 1 |
| AT4G22320 | unknown, similar to At5g55210 | 1 | / | 1 |

TAP total gives the total number of time that an interactor was co-purified;
C-GS and N-GS refers to whether a C or N terminal GS-tag was used in the experiment.

### Example 5

### Identification of SWI3C Interactors

SWI3C interactors were identified according to the methods described above. The results are summarized in Table 5. There is a strong similarity in interactors identified with ARP4 and with SWI3C; all AN3 interacting proteins that do interact with ARP4 are also interacting with SWI3C. The interaction between ARP4 and SWI3C is confirmed in both experiments.

TABLE 5
Interactors of SWI3C, identified by TAP analysis on
cell suspension cultures.
Table 5. 35S-SWI3C (5 experiments)

| | | TAP | | |
|---|---|---|---|---|
| AT number | Protein name | total | C-GS | N-GS |
| AT1G21700 | SWI3C | 5 | 2 | 3 |
| AT5G14170 | Swp73B | 5 | 2 | 3 |
| AT2G46020 | BRM | 5 | 2 | 3 |
| AT3G60830 | ARP7 | 5 | 2 | 3 |
| AT1G18450 | ARP4 | 5 | 2 | 3 |
| AT3G01890 | Swp73A | 5 | 2 | 3 |
| AT5G17510 | expressed protein, similar to | 5 | 2 | 3 |
| | At3g03460 | | | |
| AT1G20670 | DNA-binding bromodomain- | 5 | 2 | 3 |
| | containing protein, similar to | | | |
| | At1g76380 | | | |
| AT1G76380 | DNA-binding bromodomain- | 4 | 2 | 2 |
| | containing protein, similar to | | | |
| | At1g20670 | | | |
| AT5G55210 | expressed protein, similar to | 4 | 2 | 2 |
| | At4g22320 | | | |
| AT3G03460 | unknown, similar to At5g17510 | 1 | / | 1 |
| AT4G22320 | unknown, similar to At5g55210 | 1 | 1 | / |
| AT1G11840 | GLX1 (GLYOXALASE I | 1 | 1 | / |
| | HOMOLOG) | | | |
| AT4G14880 | OASA1 (O-ACETYLSERINE | 1 | 1 | / |
| | (THIOL) LYASE (OAS-TL) | | | |
| | ISOFORM A1) | | | |
| AT4G04740 | CPK23 (calcium-dependent protein | 1 | / | 1 |
| | kinase 23) | | | |

TAP total gives the total number of time that an interactor was copurified;
C-GS and N-GS refers to whether a C or N terminal GS-tag was used in the experiment.

### Example 6

### Overexpression Studies of SWI3C

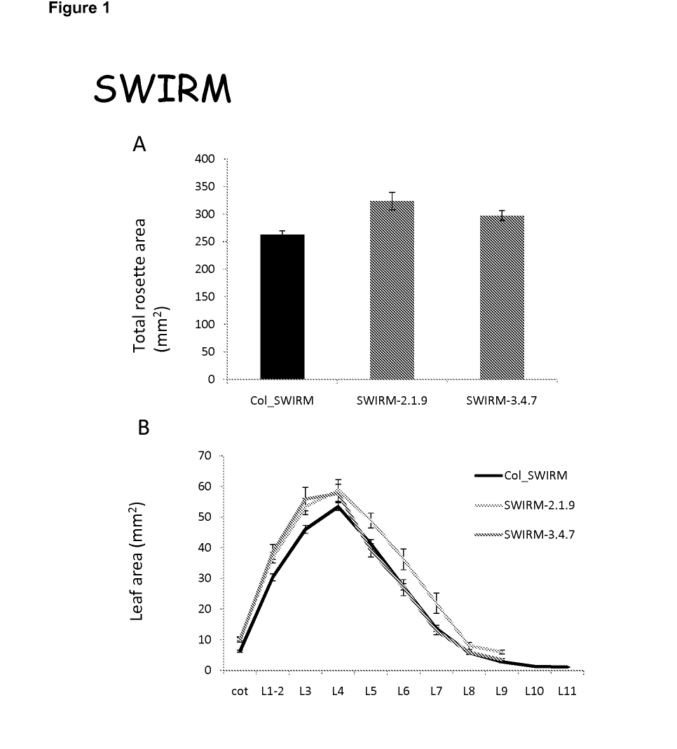

Several SWI3C overexpressing lines of Arabidopsis thaliana (Ecotype Columbia) were isolated and analyzed for growth characteristics. Amongst the 13 SWI3C overexpressing lines that were analyzed, 8 showed clearly development of bigger leaves; the bigger leaves are correlated with a higher expression of SWI3C. The detailed analysis of two SWI3C overexpressing lines is shown in FIG. 1, demonstrating that in the overexpressing lines both the individual leaves as well as the total rosette area is larger than for the control.

### REFERENCES

Altschul, S. F., Madden, T. L., Schäffer, A. A., Zhang, J., Zhang, Z., Miller, W. and Lipman, D. L. (1997), Gapped BLAST and PSI-BLAST: a new generation of protein database search programs, Nucleic Acids Res. 25, 3389-3402.
Altschul, S. F., Wootton, J. C., Gertz, E. M., Agarwala, R., Morgulis, A., Schäffer, A. A. and Yu, Y. K. (2005). Protein database searches using compositionally adjusted substitution matrices, FEBS J. 272, 5101-5109.
Bürckstümmer T, Bennett K L, Preradovic A, Schütze G, Hantschel O, Superti-Firga G, Bauch A (2006) An efficient tandem affinity purification procedure for interaction proteomics in mammalian cells. Nat Methods 3: 1013-1019.
Clark J, Rocques P J, Crew A J, Gill S, Shipley J, Chan A M, Guterson B A, Cooper C S (1994) Identification of novel genes, SYT and SSX, in the t(X;19)(p11.2;q11.2) translocation found in human synovial sarcoma. Nat Genet 7: 502-508 de Bruijn D R, Baats E, Zechner U, de Leeuw B, Balemans M, Olde Weghuis D, Hirning-Folz U, Geurts van Kessel A G (1996) Isolation and characterization of the mouse homolog of SYT, a gene implicated in the development of human synovial sarcomas. Oncogene 13: 643-648
Horiguchi G, Kim G-T, Tsukaya H (2005) The transcription factor AtGRF5 and the transcription coactivator AN3 regulate cell proliferation in leaf primordial of Arabidopsis thaliana. Plant J 43: 68-78
Hurtado L, Farrona S, Reyes J C (2006) The putative SWI/SNF complex subunit BRAHMA activates flower homeotic genes in Arabidopsis thaliana. Plant Mol Biol 62: 291-304
Ishida M, Tanaka S, Ohki M, Ohta T (2004) Transcriptional co-activator activity of SYT is negatively regulated by BRM and Brg1. Genes Cells 9: 419-428
Jerzmanowski A (2007) SWI/SNF chromatin remodeling and linker histones in plants. Biochim Biophys Acta. 1769: 330-345.
Kim J H, Choi D, Kende H (2003) The AtGRF family of putative transcription factors is involved in leaf and cotyledon growth in Arabidopsis. Plant J 36: 94-104
Kim J H, Kende H (2004) A transcriptional coactivator, AtGIF1, is involved in regulating leaf growth and morphology in Arabidopsis. Proc Natl Acad Sci USA 1001: 13374-13379
Kwon C S, Hibara K-I, Pfluger J, Bezhani S, Metha H, Aida M, Tasaka M, Wagner D (2006) A role for chromatin remodeling in regulation of CUC gene expression in the Arabidopsis cotyledon boundary. Development 133: 3223-3230
Meagher R B, Deal R B, Kandasamy M K, McKinney E C (2005) Nuclear actin-related proteins as epigenetic regulators of development. Plant Physiol 139: 1579-1585
Sarnowski T J, Rios G, Jasik J, Swiezewski S, Kaczanowski S, Li Y, Kwiatkowska A, Pawlikowska K, Kozbial M, Koncz C, Jerzmanowski A (2005) SWI3 subunits of putative SWI/SNF chromatin-remodeling complexes play distinct roles during Arabidopsis development. Plant Cell 17: 2454-2472
Thaete C, Brett D, Monaghan P, Whitehouse S, Rennie G, Rayner E, Cooper C S, Goodwin G (1999) Functional domains of the SYT and SYT-SSX synovial sarcoma translocation proteins and co-localization with the SNF protein BRM in the nucleus. Hum Mol Genet 8: 585-591
Van Camp W (2005) Yield enhancement genes: seeds for growth. Curr Opin Biotech 16: 147-153
Van Leene J, Stals H, Eeckhout D, Persiau G, Van De Slijke E, Van Isterdael G, De Clercq A, Bonnet E, Laukens K, Remmerie N, Henderickx K, De Vijlder T, Abdelkrim A, Pharazyn A, Van Onckelen H, Inzé D, Witters E, De Jaeger G (2007) A tandem affinity purification-based technology platform to study the cell cycle interactome in Arabidopsis thaliana. Mol Cell Proteomics 6: 1226-1238
Wagner D, Meyerowitz E M (2002) SPLAYED, a novel SWI/SNF ATPase homolog, controls reproductive development in Arabidopsis. Current Biol 12: 85-94

1. An isolated AN3-protein free protein complex, comprising at least a plant variant of a SWI/SNF3 subunit, said variant capable of interacting with AN3p, and one or more of the proteins interacting with said variant SWI/SNF3 subunit.

2. The isolated AN3-protein free protein complex of claim 1, whereby said SWI/SNF3 subunit is selected from the group consisting of proteins encoded by AT1G18450 (ARP4), AT3G60830 (ARP7), AT5G14170 (S 73B) and AT1G21700 (SWI3C), or a variant thereof.

3. The isolated AN3-protein free protein complex of claim 2, comprising at least ARP4p and a protein selected from the group consisting of proteins encoded by AT5G45600, AT1G76380, AT3G01890, AT5G26360, AT5G14240, AT1G47128, AT2G27100, AT5G55040, AT3G03460 and AT1G54390, or a variant thereof.

4. The isolated AN3-protein free protein complex of claim 2, comprising at least ARP7p and a protein selected from the group consisting of proteins encoded by AT3G20050, AT5G14240, AT4G22320, AT5G26360, AT3G02530, AT3G18190, AT3G03960, AT3G08580, AT4G14880 and AT1G07820, or a variant thereof.

5. The isolated AN3-protein free protein complex of claim 2, comprising at least Swp73Bp and a protein selected from the group consisting of proteins encoded by AT2G47620, AT2G33610, AT3G17590, AT4G34430, AT1G32730, AT3G22990, AT1G06500, AT1G47128, AT3G18380, AT3G06010, AT1G58025, AT5G03290, AT5G55040, AT3G50000, AT4G28520, AT5G44120 and AT4G22320, or a variant thereof.

6. The isolated AN3-protein free protein complex of claim 2, comprising at least SWI3Cp and a protein selected from the group consisting of proteins encoded by AT3G01890, AT1G76380, AT3G03460, AT4G22320, AT1G11840, AT4G14880 and AT4G04740, or a variant thereof.

7. A method for modulating plant growth and/or plant yield, comprising utilizing the isolated AN3-protein free protein complex of claim 1.

8. The method of claim 7, whereby said modulation of plant growth and/or plant yield is an increase of plant growth and/or plant yield.

9. A method to promote the complex formation of the protein complex of claim 1, comprising overexpressing at least one protein of said protein complex.

10. A method to inhibit complex formation of the protein complex of claim 1, comprising downregulating the expression of at least one protein of said protein complex.


SEQUENCE LISTING (59 sequences)

SEQ ID NO: 1 - protein, 210 aa, Arabidopsis thaliana
  MISC_FEATURE (1)..(210): AT5G28640
MQQHLMQMQPMMAGYYPSNVTSDHIQQYLDENKSLILKIVESQNSGKLSECAENQARLQRNLMYLAAIADSQPQPPSVHSQYGSAGGGMIQGEGGSHYLQQQQATQQQQMTQQSLMAARSSMLYAQQQQQQQPYATLQHQQLHHSQLGMSSSSGGGGSSGLHILQGEAGGFHDFGRGKPEMGSGGGGEGRGGSSGDGGETLYLKSSDDGN

SEQ ID NO: 2 - protein, 535 aa, Arabidopsis thaliana
  misc_feature (1)..(535): AT4G16143
MSLRPNAKTEVRRNRYKVAVDAEEGRRRREDNMVEIRKSKREESLQKKRREGLQANQLPQFAPSPVPASSTVEKKLESLPAMVGGVWSDDRSLQLEATTQFRKLLSIERSPPIEEVIDAGVVPRFVEFLTREDYPQLQFEAAWALTNIASGTSENTKVVIEHGAVPIFVQLLASQSDDVREQAVWALGNVAGDSPRCRDLVLGQGALIPLLSQLNEHAKLSMLRNATWTLSNFCRGKPQPPFDQVRPALPALERLIHSTDEEVLTDACWALSYLSDGTNDKIQSVIEAGVVPRLVELLQHQSPSVLIPALRSIGNIVTGDDLQTQCVISHGALLSLLSLLTHNHKKSIKKEACWTISNITAGNRDQIQAVCEAGLICPLVNLLQNAEFDIKKEAAWAISNATSGGSPDQIKYMVEQGVVKPLCDLLVCPDPRIITVCLEGLENILKVGEAEKVTGNTGDVNFYAQLIDDAEGLEKIENLQSHDNSEIYEKAVKILETYWLEEEDETLPPGDPSAQGFQFGGGNDAAVPPGGFNFQ

SEQ ID NO: 3 - protein, 532 aa, Arabidopsis thaliana
  misc_feature (1)..(532): AT3G06720
MSLRPNAKTEVRRNRYKVAVDAEEGRRRREDNMVEIRKSKREESLMKKRREGMQALQGFPSASAASVDKKLDSLKDMVAGVWSDDPALQLESTTQFRKLLSIERSPPIEEVISAGVVPRFVEFLKKEDYPAIQFEAAWALTNIASGTSDHTKVVIDHNAVPIFVQLLASPSDDVREQAVWALGNVAGDSPRCRDLVLGCGALLPLLNQLNEHAKLSMLRNATWTLSNFCRGKPQPHFDQVKPALPALERLIHSDDEEVLTDACWALSYLSDGTNDKIQTVIQAGVVPKLVELLLHHSPSVLIPALRTVGNIVTGDDIQTQCVINSGALPCLANLLTQNHKKSIKKEACWTISNITAGNKDQIQTVVEANLISPLVSLLQNAEFDIKKEAAWAISNATSGGSHDQIKYLVEQGCIKPLCDLLVCPDPRIITVCLEGLENILKVGEAEKNLGHTGDMNYYAQLIDDAEGLEKIENLQSHDNNEIYEKAVKILETYWLEEEDDETQQPPGVDGSQAGFQFGGNQAPVPSGGFNFS

SEQ ID NO: 4 - protein, 870 aa, Arabidopsis thaliana
  misc_feature (1)..(870): AT5G53480
MAMEVTQLLINAQSIDGTVRKHAEESLKQFQEQNLAGFLLSLAGELANDEKPVDSRKLAGLVLKNALDAKEQHRKYELVQRWLALDMSTKSQIRAFLLKTLSAPVPDVRSTASQVIAKVAGIELPQKQWPELIVSLLSNIHQLPAHVKQATLETLGYLCEEVSPDVVEQEHVNKILTAVVQGMNAAEGNTDVRLAATRALYMALGFAQANFNNDMERDYIMRVVCEATLSPEVKIRQAAFECLVSIASTYYEKLAHYMQDIFNITAKAVREDDESVALQAIEFWSSICDEEIDILEEYGGEFAGDSDVPCFYFTKQALPGLVPLLLETLLKQEEDQDLDEGAWNIAMAGGTCLGLVARAVGDDIVPHVMPFIEEKISKPDWREREAATYAFGSILEGPSADKLMAIVNAALTFMLNALTNDPSNHVKDTTAWTLGRIFEFLHGSTIETPIINQANCQQIITVLIQSMNDAPNVAEKACGALYFLAQGYEDIGPSSPLTPFFQEIIKSLLAVAHREDATESRLRTAAYEALNEVVRCSTDETSTMVLQLVPVIMMELHNTLEGEKLSLDEREKQNELQGLLCGCLQVIIQKLGSEPTKSKFMEYADQMMGLFLRVFGCRSATAHEEAMLAIGALAYAAGPNFAKYMPEFYKYLEMGLQNFEEYQVCAVTVGVVGDVCRALEDKILPYCDGIMTQLLKDLSSNQLHRSVKPPIFSCFGDIALAIGEDFDKYWRYSMPMLQSAAELSAHSAGADDEMTEYTNSLRNGILEAYSGIFQGFKNSAKTQLLIPFAPHILQFLDSIYMEKDMDEVVMKTAIGVLGDLADTLGSHVGGLIQQSVSSKEFLNECLSSEDHTIKEAAEWAKHAITRAISV

SEQ ID NO: 5 - protein, 538 aa, Arabidopsis thaliana
  misc_feature (1)..(538): AT1G09270
MSLRPSTRAELRKKIYKTGVDADEARRRREDNLVEIRKNKREDSLLKKRREGMMLQQQLPLGAGLDGPQTAAAVEKRLEGIPMMVQGVYSDDPQAQLEATTQFRKLLSIERSPPIDEVIKAGVIPRFVEFLGRHDHPQLQFEAAWALTNVASGTSDHTRVVIEQGAVPIFVKLLTSASDDVREQAVWALGNVAGDSPNCRNLVLNYGALEPLLAQLNENSKLSMLRNATWTLSNFCRGKPPTPFEQVKPALPILRQLIYLNDEEVLTDACWALSYLSDGPNDKIQAVIEAGVCPRLVELLGHQSPTVLIPALRTVGNIVTGDDSQTQFIIESGVLPHLYNLLTQNHKKSIKKEACWTISNITAGNKLQIEAVVGAGIILPLVHLLQNAEFDIKKEAAWAISNATSGGSHEQIQYLVTQGCIKPLCDLLICPDPRIVTVCLEGLENILKVGEADKEMGLNSGVNLYAQIIEESDGLDKVENLQSHDNNEIYEKAVKILERYWAEEEEEQILQDGGNDNSQQAFNFGNNPAAPVGGFKFA

SEQ ID NO: 6 - protein, 3574 aa, Arabidopsis thaliana
  misc_feature (1)..(3574): AT2G28290
MTSSSHNIELEAAKFLHKLIQDSKDEPAKLATKLYVILQHMKTSGKENTMPYQVISRAMDTVVNQHGLDIEALKSSCLPHPGGTQTEDSGSAHLAGSSQAVGVSNEGKATLVENEMTKYDAFTSGRQLGGSNSASQTFYQGSGTQSNRSFDRESPSNLDSTSGISQPHNRSETMNQRDVKSSGKRKRGESSLSWDQNMDNSQIFDSHKIDDQTGEVSKIEMPGNSGDIRNLHVGLSSDAFTTPQCGWQSSEATAIRPAIHKEPGNNVAGEGFLPSGSPFREQQLKQLRAQCLVFLALRNGLVPKKLHVEIALRNTFREEDGFRGELFDPKGRTHTSSDLGGIPDVSALLSRTDNPTGRLDEMDFSSKETERSRLGEKSFANTVFSDGQKLLASRIPSSQAQTQVAVSHSQLTFSPGLTKNTPSEMVGWTGVIKTNDLSTSAVQLDEFHSSDEEEGNLQPSPKYTMSQKWIMGRQNKRLLVDRSWSLKQQKADQAIGSRFNELKESVSLSDDISAKTKSVIELKKLQLLNLQRRLRSEFVYNFFKPIATDVEHLKSYKKHKHGRRIKQLEKYEQKMKEERQRRIRERQKEFFGGLEVHKEKLEDLFKVRRERLKGFNRYAKEFHKRKERLHREKIDKIQREKINLLKINDVEGYLRMVQDAKSDRVKQLLKETEKYLQKLGSKLKEAKLLTSRFENEADETRTSNATDDETLIENEDESDQAKHYLESNEKYYLMAHSIKENINEQPSSLVGGKLREYQMNGLRWLVSLYNNHLNGILADEMGLGKTVQVISLICYLMETKNDRGPFLVVVPSSVLPGWQSEINFWAPSIHKIVYCGTPDERRKLFKEQIVHQKFNVLLTTYEYLMNKHDRPKLSKIHWHYIIIDEGHRIKNASCKLNADLKHYVSSHRLLLTGTPLQNNLEELWALLNFLLPNIFNSSEDFSQWFNKPFQSNGESSAEEALLSEEENLLIINRLHQVLRPFVLRRLKHKVENELPEKIERLIRCEASAYQKLLMKRVEDNLGSIGNAKSRAVHNSVMELRNICNHPYLSQLHSEEVNNIIPKHFLPPIVRLCGKLEMLDRMLPKLKATDHRVLFFSTMTRLLDVMEDYLTLKGYKYLRLDGQTSGGDRGALIDGFNKSGSPFFIFLLSIRAGGVGVNLQAADTVILFDTDWNPQVDLQAQARAHRIGQKKDVLVLRFETVNSVEEQVRASAEHKLGVANQSITAGFFDNNTSAEDRKEYLESLLRESKKEEDAPVLDDDALNDLIARRESEIDIFESIDKQRKENEMETWNTLVHGPGSDSFAHIPSIPSRLVTEDDLKLLYETMKLNDVPMVAKESTVGMKRKDGSMGGLDTHQYGRGKRAREVRSYEEKLTEEEFEKLCQTESPDSPQGKGEGSERSLANDTSNIPVENSSDTLLPTSPTQAITVQPMEPVRPQSHTLKEETQPIKRGRGRPKRTDKALTPVSLSAVSRTQATGNAISSAATGLDFVSSDKRLEAASHPTSSLALTSPDLSGPPGFQSLPASPAPTPIRGRGRGRSRGRGAGRGRRVEGVLHGSNSSITQRTETATSLASDAEATKFALPRSASEIVSRVPKANEGSTSNPDQVSPVHSATTALRSDKAADKDLDAPPGFDSGSHVQTLNVLENSSERKAFAVKKRPLIQGVSSQHPGPNKQPLDLPVSTSSTLLGGGPVQNQNAVSSVCDGSKSPSEGRTYTALQGVTTAPSDATLPMSSQPSDATLPMSSQPVGSTVEAQEANVPSLPAALPAKRRVRNLPSRGETPKRQGKRRGQPLPATDASSARSTGLTPQIEVKVGNLSGTKAKFDAVAKEQPHFSQSVAPDIHSSGSLSQEIRRDTSGTGGSARKQTADVTDVARVMKEIFSETSLLKHKVGEPSATTRTNVPDAQSPGEMNLHTVETHKAEDSSGLKNQEALYNLSKADKLVSDIPHPVPGDLTTSGSVANKDVDIGSSKVAAENELVKIPGGDVDSSVIQLSLGNTLTAKSSLEKCTADQLLGEKLSQEGETTPASDGETCHLAEETASSLSYVRSEPTASASTTAEPLPTDKLEKNISFQDEVKTLNGDKREAILLSSEEQTNVNSKIETNSEELQASRTDEVPHVDGKSVDVANQTVKEDEAKHSVEIQSSMLEPDELPNAGQKGHSSIDLQPLVLVTSNENAMSLDDKDYDPISKSADIEQDPEESVFVQGVGRPKVGTADTQMEDTNDAKLLVGCSVESEEKEKTLQSLIPGDDADTEQDPEESVSDQRPKVGSAYTQMEDTDEAKLLMGCSVESEEKEKTLQSHIPGDDADTEKNPEESVSVQGVDRPKVGTTDTQMEDTNDAKLLVGCSVASEEKEKTLQSHIPGDDADTEQNPEESVSVQGVNRPKVGNANTQMEDTDEAKVLVGCSVESEEKEKTLQSHIPGDDADTEQNPEESVSNFDRPKDGTADTHMEDIDDAKLLVGCSVESEEKEKSLQSHMPSDDAVLHAPFENTKDSKGDDLHGESLVSCPTMEVMEQKGFESETHARTDSGGIDRGNEVSENMSDGVKMNISSVQVPDASHDLNVSQDQTDIPLVGGIDPEHVQENVDVPASPHGAAPNIVIFQSEGHLSPSILPDDVAGQLESMSNDEKTNISSEQVPDVSHDLKVSQDQTDIPPVGGIVPENLQEIVDVPASPHGVVPDVVVSQSEEIQSPSILPDDVPGQPDDGNCEKMDTMQNNTSIDIGITSGKTCQPSSSTQPEDENRNSLSHCEPSEVVEQRDSRDQVCIGSVESQVEISSAILENRSADIQPPQSILVDQKDIEESKEPGIESADVSLHQLADIQAEPSNLVDQMDIEESKEPGTESADVSLHQLADIQPGPSILVDQMDTEKSKEPGTESADVSLHQLADIQPGPSILVDQMDTEKSKEPGTESADVSLHQLADIQPGPSILVDQMDTEEFKNPDVSLHQLADIEPSLSISAVQKNIEDKDQSHVETAGSELVDVSAECSTEPQVQLPPSSEPVGDMHVHLGASKSEIVAEGTDFSSSLPKTEEENAKSQLADTEPSSSLTAVQKNIEDQVETAGCEFVVVSTGCSTEPQVQLPPSAEPVVAEGTEFPSSLLMTGVDNSSHLMTGVDNAKTHLADVVPSSSPTTMEKNIEAQDQDQVTTGGCGLVDVLTECSSEPQLQLPPSAEPVISEGTELATLPLTEEENADSQLANIEPSSSPSVVEKNIEAQDQDQVKTAGCELVSTGCSSEPQVHLPPSAEPDGDIHVHLKETEKSESMVVVGEGTAFPSSLPVTEEGNAESQLADTEPFTSPTVVEKNIKDQEQVETTGCGLVDDSTGCSSEPQVQLPPSAEPMEGTHMHLEETKKSETVVTEIQLADIDPSFSLIVVQTNIEDQDQIETGGCDLINVPSGCSTEPQIQLSSSAEPEEGMHIHLEAAMNSETVVTEGSELPSSLPMTEDENADGQLAEVEPSVSLTVEQTNIEEKDHIETAECELVDVSPGCSSQPEVKFPPSPDAVGGMDVHLETVVTEDTDSNSSLPKTEEKDAENPSDRLDGESDGTTVATVEGTCVESNSLVAEESNIEVPKDNEDV

SEQ ID NO: 7 - protein, 363 aa, Arabidopsis thaliana
  misc_feature (1)..(363): AT3G60830
MEALVVDAGSKFLKAGAAIPDQSPAMIIPSQMKRMVDDGSSSADNPTTVFEDVTLDPIERGLIRDWDAMEDLLRYVVYTGLGWEEGNEGNILFTDPLCTPKAIREQLVQLMFETFNVSGFYASEQAVLSLYAVGRISGCTVDIGHGKIDIAPVLEGAVQHIASKRFELGGTELTKLFAQELGKTNPSMNLSMSDVEKLKEQYANCAEDEIAYKKTQNCEIEQHTLPDGQVISIGRERYSVGEALFQPSILGLEEHGIVEQLVRIISTVSSENHRQLLENTVLCGGTTSMTGFESRFQKEANLCSSAIRPTLVKPPEYMPENLGMYSAWVGGAILAKVVFPQNQHVTKADYDETGPSVVHRKCF

SEQ ID NO: 8 - protein, 2193 aa, Arabidopsis thaliana
  misc_feature (1)..(2193): AT2G46020
MQSGGSGGGPARNPAMGPAGRTASTSSAASPSSSSSSVQQQQQQQQQQQQQQQLASRQQQQQHRNSDTNENMFAYQPGGVQGMMGGGNFASSPGSMQMPQQSRNFFESPQQQQQQQQQGSSTQEGQQNFNPMQQAYIQFAMQAQHQKAQQQARMGMVGSSSVGKDQDARMGMLNMQDLNPSSQPQASSSKPSGDQFARGERQTESSSQQRNETKSHPQQQVGTGQLMPGNMIRPMQAPQAQQLVNNMGNNQLAFAQQWQAMQAWARERNIDLSHPANASQMAHILQARMAAQQKAGEGNVASQSPSIPISSQPASSSVVPGENSPHANSASDISGQSGSAKARHALSTGSFASTSSPRMVNPAMNPFSGQGRENPMYPRHLVQPTNGMPSGNPLQTSANETPVLDQNASTKKSLGPAEHLQMQQPRQLNTPTPNLVAPSDTGPLSNSSLQSGQGTQQAQQRSGFTKQQLHVLKAQILAFRRLKKGEGSLPPELLQAISPPPLELQTQRQISPAIGKVQDRSSDKTGEDQARSLECGKESQAAASSNGPIFSKEEDNVGDTEVALTTGHSQLFQNLGKEATSTDVATKEEQQTDVFPVKSDQGADSSTQKNPRSDSTADKGKAVASDGSQSKVPPQANSPQPPKDTASARKYYGPLFDFPFFTRKLDSYGSATANANNNLTLAYDIKDLICEEGAEFLSKKRTDSLKKINGLLAKNLERKRIRPDLVLRLQIEEKKLRLSDLQSRVREEVDRQQQEIMSMPDRPYRKFVRLCERQRLEMNRQVLANQKAVREKQLKTIFQWRKKLLEAHWAIRDARTARNRGVAKYHEKMLREFSKRKDDGRNKRMEALKNNDVERYREMLLEQQTNMPGDAAERYAVLSSFLTQTEDYLHKLGGKITATKNQQEVEEAANAAAVAARLQGLSEEEVRAAATCAREEVVIRNRFTEMNAPKENSSVNKYYTLAHAVNEVVVRQPSMLQAGTLRDYQLVGLQWMLSLYNNKLNGILADEMGLGKTVQVMALIAYLMEFKGNYGPHLIIVPNAVLVNWKSELHTWLPSVSCIYYVGTKDQRSKLFSQEVCAMKFNVLVTTYEFIMYDRSKLSKVDWKYIIIDEAQRMKDRESVLARDLDRYRCQRRLLLTGTPLQNDLKELWSLLNLLLPDVFDNRKAFHDWFAQPFQKEGPAHNIEDDWLETEKKVIVIHRLHQILEPFMLRRRVEDVEGSLPAKVSVVLRCRMSAIQSAVYDWIKATGTLRVDPDDEKLRAQKNPIYQAKIYRTLNNRCMELRKACNHPLLNYPYFNDFSKDFLVRSCGKLWILDRILIKLQRTGHRVLLFSTMTKLLDILEEYLQWRRLVYRRIDGTTSLEDRESAIVDFNDPDTDCFIFLLSIRAAGRGLNLQTADTVVIYDPDPNPKNEEQAVARAHRIGQTREVKVIYMEAVVEKLSSHQKEDELRSGGSVDLEDDMAGKDRYIGSIEGLIRNNIQQYKIDMADEVINAGRFDQRTTHEERRMTLETLLHDEERYQETVHDVPSLHEVNRMIARSEEEVELFDQMDEEFDWTEEMTNHEQVPKWLRASTREVNATVADLSKKPSKNMLSSSNLIVQPGGPGGERKRGRPKSKKINYKEIEDDIAGYSEESSEERNIDSGNEEEGDIRQFDDDELTGALGDHQTNKGEFDGENPVCGYDYPPGSGSYKKNPPRDDAGSSGSSPESHRSKEMASPVSSQKFGSLSALDTRPGSVSKRLLDDLEEGEIAASGDSHIDLQRSGSWAHDRDEGDEEQVLQPTIKRKRSIRLRPRQTAERVDGSEMPAAQPLQVDRSYRSKLRTVVDSHSSRQDQSDSSSRLRSVPAKKVASTSKLHVSSPKSGRLNATQLTVEDNAEASRETWDGTSPISSSNAGARMSHIIQKRCKIVISKLQRRIDKEGQQIVPMLTNLWKRIQNGYAAGGVNNLLELREIDHRVERLEYAGVMELASDVQLMLRGAMQFYGFSHEVRSEAKKVHNLFFDLLKMSFPDTDFREARNALSFSGSAPTLVSTPTPRGAGISQGKRQKLVNEPETEPSSPQRSQQRENSRIRVQIPQKETKLGGTTSHTDESPILAHPGELVICKKKRKDREKSGPKTRTGGSSSPVSPPPAMIGRGLRSPVSGGVPRETRLAQQQRWPNQPTHPNNSGAAGDSVGWANPVKRLRTDSGKRRPSHL

SEQ ID NO: 9 - protein, 441 aa, Arabidopsis thaliana
  misc_feature (1)..(441): AT1G18450
MYGGDEVSAIVVDLGSHTCKAGYAGEDAPKAVFPSVIGAVDGVEAMDVDVDSTKTNSNSEDSKTESEKEKSKRKLYVGSQAMSYRRDHMEVLSPIKDGIVSDWDLVDNIWEHAFKSCLMIDPTEHPMLLAEPPLNTQQQREKAAELMFEKYKVPALFMAKNPVLTSFATGRATSLVVDCGGGSTTISPVHDGYVLQKAVVSSPLGGEFLTDCLLKSLESKGIKIRPRYSFKRKEVRAGEFQVEDVDIPDTTESYKLFCQRMIVGDIKDSICRVPDTPYDDKSYSNIPTTSYELPDGQTLEIGADRFKVPDVMFNPSIVQTIPGMEKYAEMIPSVRGLPHMVMESINKCDVDIRRELYSSILLAGGTSSMQQLKERLEKDLIEESPHSARVKVLASGNTTERRFSVWIGGSILASLGSFQQMWFSKSEYEEHGASYIQRKCP

SEQ ID NO: 10 - protein, 807 aa, Arabidopsis thaliana
  misc_feature (1)..(807): AT1G21700
MPASEDRRGKWKRKKRGGLSAARKPKQEEEDMEEEDEENNNNNNEEMDDVENADELQQNGGATPDPGLGIGEVVEDSGSRISDFPAVVKRVVIRPHASVMAVVAAERAGLIGETRGQGSLPALENISFGQLQALSTVPADSLDLERSDGSSSAYVISPPPIMDGEGVVKRFGDLVHVLPMHSDWFAPNTVDRLERQVVPQFFSGKSPNHTPESYMEFRNAIVSKYVENPEKTLTISDCQGLVDGVDIEDFARVFRFLDHWGIINYCATAQSHPGPLRDVSDVREDTNGEVNVPSAALTSIDSLIKFDKPNCRHKGGEVYSSLPSLDGDSPDLDIRIREHLCDSHCNHCSRPLPTVYFQSQKKGDILLCCDCFHHGRFVVGHSCLDFVRVDPMKFYGDQDGDNWTDQETLLLLEAVELYNENWVQIADHVGSKSKAQCILHFLRLPVEDGLLDNVEVSGVTNTENPTNGYDHKGTDSNGDLPGYSEQGSDTEIKLPFVKSPNPVMALVAFLASAVGPRVAASCAHESLSVLSEDDRMKSEGMQGKEASLLDGENQQQDGAHKTSSQNGAEAQTPLPQDKVMAAFRAGLSAAATKAKLFADHEEREIQRLSANIVNHQLKRMELKLKQFAEIETLLMKECEQVEKTRQRFSAERARMLSARFGSPGGISPQTNNLQGMSLSTGGNNINSLMHQQHQQQQASATSQPSIIPGFSNNPQVQAQMHFMARQQQQQQQQQQQQQQAFSFGPRLPLNAIQTNAGSTASPNVMFGNNQLNNPAAAGAASINQPSFSHPMVRSSTGSGSGSGLGLN

SEQ ID NO: 11 - protein, 458 aa, Arabidopsis thaliana
  misc_feature (1)..(458): AT5G14170
MSGNNELNALKGPQSSLPWPFGMASASFAPSHMAPPNFQPQFQLSQDQGISAPGRRFPHKPPIGGPPAVPPSMELTPASRKKKHKLPDKSSLQERVAAVLPESALYTQLLEFESRVDAALFRKKVDIQDSLKNPPSIQKTLRIYVFNTFSNQIPGPDPPTWTLRIFGRVLDPDHTGGLVQNSNPLYPKFSSFFKTLKISLDQSLYPENHLIEWKRDRSPAPLEGFEIKRIGCQEFAATILLEMNYVPEKFKTSPALMQVLGIEVDTRPRIIAAIWHYVKVRKLQNPNDPSFFNCDAALHSVFGEEKMKFTMLSHKISQHLSPPPPIQLVHKIKLSGNNPAISACYDVLVDIPAPVQTELSNLLANPEKNKEIEACDEAICGAIRKIHEHRRRRAFFLGFSQSPVEFTNALMESQTKDLKLVAGEASRNAEKEGRSEFFNQPWVEDAAIRYLNRKPAGG

SEQ ID NO: 12 - protein, 1058 aa, Arabidopsis thaliana
  misc_feature (1)..(1058): AT4G17330
MISAFGGADGGKGSWEKSWRTCVVRAQKSLVATPETPLQSRPGKTETPSAGHTNSKESSGTNPMIPLSSPLWSLSTSVDTLQSSSVQRGSAATHQPLLSASHAHQTPPTQNIVGHNTPWMSPLPFRNAWLASQQTSGFDVGSRFPVYPITDPVKLTPMKESSMTLSGAKHVQSGTSSNVSKVTPTLEPTSTVVAPAQHSTRVKSRKRKKMPVSVESGPNILNSLKQTELAASPLVPFTPTPANLGYNAGTLPSVVSMTAVPMDLVSTFPGKKIKSSFPSPIFGGNLVREVKQRSVLSEDTIEKLKEAKMHAEDASALATAAVSHSEYVWKQIEQQSHAGLQPETQDRLASAAVAIAAAAAVAKAAAAAANVAANAALQAKLMAEEASLPNASDQGLPKSYDSILPGQGTPASILKGEGAVVNSSSVLIAAREASKKRVEAATAATKRAENMDSIVKAAELASEAVSQAGILVSMGHPPLLNKLVEAGPSNYWRQAQESQEVQPCKTVVLEKETVSTSEGTFAGPKIVQTEFGGSVNTADGVSGPVPATGKLKGQEGDKYADLAKNNDVVFEPEVGSKFSIDAQQTIKATKNEDIKEGSNVEVFKEEPGLRTAWYSANVLSLEDDKAYVLFSDLSVEQGTDKLKEWVALKGEGDQAPKIRPARSVTALPYEGTRKRRRAALGDHIWKIGDRVDSWVHDSWLEGVITEKNKKDENTVTVHFPAEEETLTIKAWNLRPSLVWKDGKWIECSSSGETISSSHEGDTPKEKRPRLGTPALVAEVKDTSMKIVDDPDLGKPPQTGVLNLGVSENTFNIGKSTREENKPDPLRMKRTGLQKQGSKVIFGVPKPGKKRKFMDVSKHYVSEASTKTQERKEPVKPVRSIVPQNSGIGSWKMPSKTISIEKQTTISRPKTFKPAPKPKEKPGATARIIPRKDSRNTTASDMESDESAENRGPGSGVSFKGTVEEQTTSSSHDTGSKNSSSLSTNKGRVAPTAGRLAKIEEDKALAENSSKTSEGMEPRRSIRRIQPTSRLLEGLQTSMMTSKIPSVSHSKSHLSQSKK

SEQ ID NO: 13 - protein, 395 aa, Arabidopsis thaliana
  misc_feature (1)..(395): AT3G15000
MATHTISRSILCRPAKSLSFLFTRSFASSAPLAKSPASSLLSRSRPLVAAFSSVFRGGLVSVKGLSTQATSSSLNDPNPNWSNRPPKETILLDGCDFEHWLVVVEPPQGEPTRDEIIDSYIKTLAQIVGSEDEARMKIYSVSTRCYYAFGALVSEDLSHKLKELSNVRWVLPDSYLDVRNKDYGGEPFIDGKAVPYDPKYHEEWIRNNARANERNRRNDRPRNNDRSRNFERRRENMAGGPPPQRPPMGGPPPPPHIGGSAPPPPHMGGSAPPPPHMGQNYGPPPPNNMGGPRHPPPYGAPPQNNMGGPRPPQNYGGTPPPNYGGAPPANNMGGAPPPNYGGGPPPQYGAVPPPQYGGAPPQNNNYQQQGSGMQQPQYQNNYPPNRDGSGNPYQG

SEQ ID NO: 14 - protein, 168 aa, Arabidopsis thaliana
  misc_feature (1)..(168): AT5G55210
MEGVGSRLSRTSSRYSGPAATAVFSGRVRKWKKKWVRVSTSSVGVFRASKSNGRNNSNNSNSPHHLLLHKWTPLTSATVTASDANGSGETEESPKRRFRYAPIAMLEHREKVISKDSEIEETEEFDTESPLPKAVELDMNLTDSDQTKEAKTGNLNLGLCLNSEGTEE

SEQ ID NO: 15 - protein, 369 aa, Arabidopsis thaliana
  misc_feature (1)..(369): AT5G17510
MEDTKPPLGGNNNPQQQQQQQQLLYQHQLQQQQRQQQMLLLQQLQKQQQQQAAMSRFPSNIDVHLRPPGLIQNRPINPPPQQNPTPNPNLGQQTPNFQQQQQQNVSSQQMMQQQQQQQQQQKLMRPLNHIELQCAYQDAWRVCHPNFKQPFSSLEDACERLLPYHVVADYEAEEDDRILDSDPTGQALSRSQQWDNNIAAKVAEFTATFEKQALAFNIITRKRAMGEFRSEERLMVEQALLQDERKALIELKAEMDREKAGREAQEAKLRMAALAQAGQSQSHAEIMARNPLRANAVGNQGSNIQLSHEMGEQGRNMNPDEMMNGWGNNSQREDKEPSEDFLNDEENENGETGEQENWREAGEFDLNSR

SEQ ID NO: 16 - protein, 566 aa, Arabidopsis thaliana
  misc_feature (1)..(566): AT2G16570
MAATTSFSSSLSLITKPNNSSYTNQPLPLFPKPFLKPPHLSLLPSPLSSPPPSLIHGVSSYFSSPSPSEDNSHTPFDYHNDEDDEKPREECGVVGIYGDPEASRLCYLALHALQHRGQEGAGIVTVSPEKVLQTITGVGLVSEVFNESKLDQLPGEFAIGHVRYSTAGASMLKNVQPFVAGYRFGSIGVAHNGNLVNYKTLRAMLEENGSIFNTSSDTEVVLHLIAISKARPFFMRIIDACEKLQGAYSMVFVTEDKLVAVRDPYGFRPLVMGRRSNGAVVFASETCALDLIEATYEREVYPGEVLVVDKDGVKSQCLMPKFEPKQCIFEHIYFSLPNSIVFGRSVYESRHVFGEILATESPVECDVVIAVPDSGVVAALGYAAKSGVPFQQGLIRSHYVGRTFIEPSQKIRDFGVKLKLSPVRGVLEGKRVVVVDDSIVRGTTSSKIVRLLREAGAKEVHMRIASPPIVASCYYGVDTPSSEELISNRLSVEEINEFIGSDSLAFLSFDTLKKHLGKDSKSFCYACFTGDYPVKPTEVKVKRGGGDFIDDGLVGSFENIEAGWVR

SEQ ID NO: 17 - protein, 566 aa, Arabidopsis thaliana; identical to SEQ ID NO: 16
  misc_feature (1)..(566): AT4G35550

SEQ ID NO: 18 - protein, 652 aa, Arabidopsis thaliana
  misc_feature (1)..(652): AT1G20670
MGEVADTMTKKKKKGRPSLLDLQKRAIKQQQQQLQQQQQQHKNNHQDDDDHHHNNNNRSGSKNPNSLNHRSKRRNPNSNDGDSPWIKDEGEDNDDDERREKKHKLLHGLNSHSHRHSPNSQSDLNLDQTPEPSFNRRNLSAAASGSDYHTGEKASKATDILQGSPVESGPTTPLPDKKLLLFILDRLQKKDTYGVYSDPVDPEELPDYFEIIKNPMDFSTLRNKLDSGAYSTLEQFERDVFLICTNAMEYNSADTVYYRQARAIQELAKKDFENLRQDSDDEEPQSQQQQQQQPKVARRGRPPKKHPEPSSIDRTASEISADALIPGDSSNKFSGAYNLRKAPPSYKFRQAESSVRINHNSETQSGWSVDWESEFPSSVVKAVNKYGMKHFNVDDNRRDTYNHLSTSTQEPSVLTTLEDELKQLIPVGLNMEYGYAKSLARYAANLGPVAWKIASRRIETVLPSGIKFGQGWVGENPAGPEEDDSQKQNILMSSGKQKCSNDLASDDHSNRILSPTASVSSAFIGNRHASSQAIEETTPPPARVLNPEIDHPSSSSHQAGLLIKTESSNGLIRGFNHNANQMLGIARQQQPNVSNEATPVSQQQGSLFPYPKQEFHRFPPDLNARLVSPNSPGSNQQTGSSSSQHPDLALQL

SEQ ID NO: 19 - protein, 1214 aa, Arabidopsis thaliana
  misc_feature (1)..(1214): AT3G55220
MYLYSLTLQQATGIVCAINGNFSGGKTQEIAVARGKILDLLRPDENGKIQTIHSVEVFGAIRSLAQFRLTGAQKDYIVVGSDSGRIVILEYNKEKNVFDKVHQETFGKSGCRRIVPGQYVAVDPKGRAVMIGACEKQKLVYVLNRDTTARLTISSPLEAHKSHTICYSLCGVDCGFDNPIFAAIELDYSEADQDPTGQAASEAQKHLTFYELDLGLNHVSRKWSNPVDNGANMLVTVPGGADGPSGVLVCAENFVIYMNQGHPDVRAVIPRRTDLPAERGVLVVSAAVHKQKTMFFFLIQTEYGDVFKVTLDHNGDHVSELKVKYFDTIPVASSICVLKLGFLFSASEFGNHGLYQFQAIGEEPDVESSSSNLMETEEGFQPVFFQPRRLKNLVRIDQVESLMPLMDMKVLNIFEEETPQIFSLCGRGPRSSLRILRPGLAITEMAVSQLPGQPSAVWTVKKNVSDEFDAYIVVSFTNATLVLSIGEQVEEVNDSGFLDTTPSLAVSLIGDDSLMQVHPNGIRHIREDGRINEWRTPGKRSIVKVGYNRLQVVIALSGGELIYFEADMTGQLMEVEKHEMSGDVACLDIAPVPEGRKRSRFLAVGSYDNTVRILSLDPDDCLQILSVQSVSSAPESLLFLEVQASIGGDDGADHPANLFLNSGLQNGVLFRTVVDMVTGQLSDSRSRFLGLKPPKLFSISVRGRSAMLCLSSRPWLGYIHRGHFHLTPLSYETLEFAAPFSSDQCAEGVVSVAGDALRIFMIDRLGETFNETVVPLRYTPRKFVLHPKRKLLVIIESDQGAFTAEEREAARKECFEAGGVGENGNGNADQMENGADDEDKEDPLSDEQYGYPKAESEKWVSCIRVLDPKTATTTCLLELQDNEAAYSVCTVNFHDKEYGTLLAVGTVKGMQFWPKKNLVAGFIHIYRFVEDGKSLELLHKTQVEGVPLALCQFQGRLLAGIGPVLRLYDLGKKRLLRKCENKLFPNTIISIQTYRDRIYVGDIQESFHYCKYRRDENQLYIFADDCVPRWLTASHHVDFDTMAGADKFGNVYFVRLPQDLSEEIEEDPTGGKIKWEQGKLNGAPNKVDEIVQFHVGDVVTCLQKASMIPGGSESIMYGTVMGSIGALHAFTSRDDVDFFSHLEMHMRQEYPPLCGRDHMAYRSAYFPVKDVIDGDLCEQFPTLPMDLQRKIADELDRTPAEILKKLEDARNKII

SEQ ID NO: 20 - protein, 1029 aa, Arabidopsis thaliana
  misc_feature (1)..(1029): AT2G46340
MPVMERVAEETVATNNIQLKARVDDVPCNKLDARHNDMVIQSETANSDCPGSSAHRNVDLTKPPPPEEAAGAKLSVEELTLGNYRIVQGSNNTNVDSPRAGKFEHLYRLARGSAFRAGDGDLDSQPRDMDQMLSRIRQQLAGAPSERQNLKPFMSRRSDQNLEAFSERLRAAGENSIMNAPALISEGVQMKTPVSSSNFSQLLLKRAMKGKGVVGKNQETPPEFVSDQDLGSKEKKLDISKSPTPHDVLPLKSSPKGNGMVSHGDGNHSKSSIGISLREFLRSSYAKREKRHGLCLFRQLVELVDSAHSKRLFLLDLRPSLFTLVPSKKLRYIGNFGKNDLESDVDEDLNRRRPVVEESSSGGRDSKKRKMDLHLNSPGNQLQATSTGRPFKRKSPVIDLNMVDARNPDSCELQQQDYIKNLSVSSVSRKQSMSTWLEEQWYTCPEEINGEDIGEKSNIYALGVLLFELLCHCESGEMHAAMMADLRHRILPPTFLSKYPKEAGFCLWLLHPEPSSRPSARDILKSELICEDDSVKSTAAAEEISELLLHFLSSLEVQKKKKASKLLQDIQTLEDDIKEAERRYSSNVSLVRSHGAIEKRVQSSPLDEHCTTSSALFVPTANTDRLMSNIRQLEDAYFFMRSQINLSSSAATARSDKTLKDRDRCSENQNENQDMSTKGKSSDQLEVFFEGLCKFARYSKFETCGTIRSGDLLNSASVVCSLSFDPDEEHIAAAGISKKIKIFDFNAFMNESVGVHYPLVEMVNKSKLSCVCWNSYIKNYLASTDYDGVVQIWDAGTGQGFSQYTEHQKRAWSVDFSPSDPTKFVSGSDDCSVKLWSINEKRSLGTIWSPANVCCVQFSSYSNHLLAFGSADYKVYCYDLRYVKTPWCTLAGHEKAVSYVKFMDSETIVSASTDNSLKLWNLNKTNSSGLSPGACSLTYKGHTNQKNFVGLSVLDGYIACGSETNEVYSYYKSLPMPMTSYKFGSVDPISGNEYFDDNGQFVSSVCWRKKSNMLVAANSTGNMKLLKLV

SEQ ID NO: 21 - protein, 633 aa, Arabidopsis thaliana
  misc_feature (1)..(633): AT5G13030
MLLRVCCPSSFFYLRPLHLFSSTAKVPFCPSLPRQFRLSPSRSSSFRRMESSPASSSSPTPVTDSSADSLAKDLQNQSLGAVDEGVKIKKKLEDFNWDHSFVKELPGDPRTDVISREVLHACYSKVSPSVEVDDPQLVAWSVSVAELLDLDPKEFERPDFPLMLSGAKPLPGAMSYAQCYGGHQFGMWAGQLGDGRAITLGEVLNSKGERWELQLKGAGRTPYSRFADGLAVLRSSIREFLCSETMHCLGIPTTRALCLLTTGQNVTRDMFYDGNPKEEPGAIVCRVSQSFLRFGSYQIHASRGKEDLDIVRKLADYAIKHHFPHIESMDRSDSLSFKTGDEDDSVVDLTSNKYAAWIVEIAERTATLVARWQGVGFTHGVLNTDNMSILGQTIDYGPFGFLDAFDPSYTPNTTDLPGRRYCFANQPDIGLWNIAQFSKTLAVAQLINQKEANYAMERYGDKFMDEYQAIMSKKLGLTKYNKEVISKLLNNMSVDKVDYTNFFRLLANVKANPNTPENELLKPLKAVLLDIGKERKEAWIKWMRSYIQEVGGSEVSDEERKARMDSVNPKYILRNYLCQSAIDAAEQGDFSEVNNLIRLMKRPYEEQPGMEKYARLPPAWAYRPGVCMLSCSS

SEQ ID NO: 22 - protein, 502 aa, Arabidopsis thaliana
  misc_feature (1)..(502): AT5G17330
MVLSHAVSESDVSVHSTFASRYVRTSLPRFKMPENSIPKEAAYQIINDELMLDGNPRLNLASFVTTWMEPECDKLIMSSINKNYVDMDEYPVTTELQNRCVNMIAHLFNAPLEEAETAVGVGTVGSSEAIMLAGLAFKRKWQNKRKAEGKPVDKPNIVTGANVQVCWEKFARYFEVELKEVKLSEGYYVMDPQQAVDMVDENTICVAAILGSTLNGEFEDVKLLNDLLVEKNKETGWDTPIHVDAASGGFIAPFLYPELEWDFRLPLVKSINVSGHKYGLVYAGIGWVIWRNKEDLPEELIFHINYLGADQPTFTLNFSKGSSQVIAQYYQLIRLGHEGYRNVMENCRENMIVLREGLEKTERFNIVSKDEGVPLVAFSLKDSSCHTEFEISDMLRRYGWIVPAYTMPPNAQHITVLRVVIREDFSRTLAERLVIDIEKVMRELDELPSRVIHKISLGQEKSESNSDNLMVTVKKSDIDKQRDIITGWKKFVADRKKTSGIC

SEQ ID NO: 23 - protein, 444 aa, Arabidopsis thaliana
  misc_feature (1)..(444): AT1G80480
MATLSTLEIATTFLSFTAPRSSAAFNYRFSSAAVSVLSRPRATTVSVRTTPSFFYSPVVRRQRFSSVSASATQTEDSDVTTKIPPDNRIPATIITGFLGSGKTTLLNHILTRDHGKRIAVIENEFGEVDIDGSLVASKSIGAEDIVMLNNGCLCCTVRGDLVRMIGELVNTKKGKFDHIVIETTGLANPAPIIQTFYAEEEIFNDVKLDGVVTLVDAKHARLHLDEVKPEGVVNEAVEQIAYADRIIVNKTDLVGEAELGSVVQRIKTINSMAQMTRTKYGNVDLDYVLGIGGFDLERIESSVNEDDKGDHHDHDHDHHHDHNHDHDHHHHDGHDHHHHSHDHTHDPGVSSVSIVCEGSLDLEKANMWLGTLLMERSEDIYRMKGLLSVHTMEERFVFQGVHDIFQGSPDRLWGREEERVNKIVFIGKNLNREELEKGFKACLI

SEQ ID NO: 24 - protein, 391 aa, Arabidopsis thaliana
  misc_feature (1)..(391): AT1G43800
MLAHKSLLSFTTQWATLMPSPSTFLASRPRGPAKISAVAAPVRPALKHQNKIHTMPPEKMEIFKSLDGWAKDQILPLLKPVDQCWQPASFLPDPALPFSEFTDQVRELRERTASLPDEYFVVLVGDMITEDALPTYQTMINTLDGVRDETGASESAWASWTRAWTAEENRHGDLLRTYLYLSGRVDMLMVERTVQHLIGSGMDPGTENNPYLGFVYTSFQERATFVSHGNTARLAKSAGDPVLARICGTIAADEKRHENAYVRIVEKLLEIDPNGAVSAVADMMRKKITMPAHLMTDGRDPMLFEHFSAVAQRLEVYTADDYADILEFLVGRWRLEKLEGLTGEGQRAQEFVCGLAQRIRRLQERADERAKKLKKTHEVCFSWIFDKQISV

SEQ ID NO: 25 - protein, 386 aa, Arabidopsis thaliana
  misc_feature (1)..(386): AT5G45620
MAALQYLESLKNTHPELGEWYNSLADLYQKKLWHQLTLKLEQFIALAVFQAGDALIQFYHNFITDFETKINLLKLAHFAVVVSRQYSEKEAAVSYLESVIEKLRATKEPRITEPIIYIETQKALFKLEQGDQKECKKILDDGKSSLDSMTDIDPSVYANFYWVSSQYHKCRQEFSDFYKSALLYLAYTSVEDLSESFKLDLAFDLSLSALLGENIYNFGELLAHPILKSLLGTNVEWLYHILQAFNHGDLVQYQELCRVHNASLIAQPALVENEKKLLEKINILCLIEIIFSRPAEDRTIPLSIIAERTKLSIEDVEHLLMKSLSVHLIEGIIDQVNGTIYVSWAQPRVLGIPQIKALRDQLDSWVDKVHTTLLSVEAETPDLVAA

SEQ ID NO: 26 - protein, 268 aa, Arabidopsis thaliana
  misc_feature (1)..(268): AT5G45600
MTNSSSSKKQAQDQPETSEPTLKSLKTKMTKSDEKQKKLKDIEISVPIVYGNVAFWLGKKASEYQSHKWAVYVRGATNEDISVVVKKVVFQLHSSFNSPTRVIEEPPFEVSESGWGEFEIAMTLHFHSDVCDKPLSLYHHLKLYPEDESGPLTMKKPVVVESYDEIVFPDPSESFLARVQNHPALTFPRLPSGYNLPAPMQVEDTGKKKRGDTKDHSLGQWFMSFSEADELLQLAAARQQVQAHIAKLRRQISLLEGQNQTVKTGSDL

SEQ ID NO: 27 - protein, 579 aa, Arabidopsis thaliana
  misc_feature (1)..(579): AT1G76380
MGEVADNNTLMKRKKKGRPSLLDLQKRALKQQQLLQRRNPNEENEEELRSSSRNPNFSNRSNRRRNSNSEDDDDERRDKKHRLLHGLNSHEGRDSSNSKSGGGDLDSDARNRRKIDGSDNTGEKASKATDILLQRSLVESTPLPDKKLLFFILDRVQKKDTYGVYSDPADPEELPDYYEIIKNPMDFTTLRKKLESGAYTTLEQFEANVFLICTNAMEYNSADTVYYRQARAMLELAKKDFGNLRQESDGEEPVSLSQQPKVVKRGRPPGSGLKKQLEQSLIDRTTSDISADAAAFTYAGDSSRLSGSYNLRKNPPSYGFRHAETSVRINHNSENQSGLLIDWEKEFPPSVVKAVNKYGMKNVDENRRDTYNQNSASLQDSSIFTLLDDNLKQLTPVGLKAEYGYARSLARYAANIGPVAWTFANVRIEKLLPTGTEFGPGWVGENPENPPQQQNLMSGKQKCSNDYASDDHHQSSRIMSPSTSVSSSIIGNIHSSHESKESVQVLNQETEINGLVRGSSGFNHKPNQMLETAGSQQGNIKQEFQRLPPDLNARLSSPNSPGSNHQAGSSQHPDLALQL

SEQ ID NO: 28 - protein, 458 aa, Arabidopsis thaliana; identical to SEQ ID NO: 11
  misc_feature (1)..(458): AT3G01890

SEQ ID NO: 29 - protein, 555 aa, Arabidopsis thaliana
  misc_feature (1)..(555): AT5G26360
MHAPVLVLSDSLKRESGSKVHHGNIQASKAVADIIRTTLGPRSMLKMLLDAGGGIVVTNDGNAILRELDVAHPAAKSMIELSRTQDEEVGDGTTSVIVLAGEMLHVAEAFLEKNYHPTVICRAYIKALEDSIAVLDKIAMSIDINDRSQVLGLVKSCIGTKFTSQFGDLIADLAIDATTTVGVDLGQGLREVDIKKYIKVEKVPGGQFEDSEVLKGVMFNKDVVAPGKMKRKIVNPRIILLDCPLEYKKGENQTNAELVREEDWEVLLKLEEEYIENICVQILKFKPDLVITEKGLSDLACHYFSKAGVSAIRRLRKTDNNRIAKACGAVIVNRPDELQESDIGTGAGLFEVKKIGDDFFSFIVDCKEPKACTVLLRGPSKDFINEVERNLQDAMSVARNIIKNPKLVPGGGATELTVSATLKQKSATIEGIEKWPYEAAAIAFEAIPRTLAQNCGVNVIRTMTALQGKHANGENAWTGIDGNTGAIADMKESKIWDSYNVKAQTFKTAIEAACMLLRIDDIVSGIKKKQAPGSGPSKPTIETEGDADNEQILPD

SEQ ID NO: 30 - protein, 256 aa, Arabidopsis thaliana
  misc_feature (1)..(256): AT5G14240
MADYHFVYKDIEGASTQWDDIQRKLGNLPEKAPAFKPPAYTPAEDESSAPKDQAWFDKKTEEELEDLEDDKDLDDDRFLEEYRKKRLSELREAAKVKRYGTVTPISGSDFVREVTQASAEDWVVVCLYKDGVAECSLLLGCLDELGSRYPATKFVKIISTDCIPNYPDCNLPTLLVYHHGAIKGTHVGLKSFGRRCTPESVALLLCQSEPVLNDGKGGDDDSSREAVMAGVRRQFIERVVKDHEDKDNDDGGYNSD

SEQ ID NO: 31 - protein, 462 aa, Arabidopsis thaliana
  misc_feature (1)..(462): AT1G47128
MGFLKPTMAILFLAMVAVSSAVDMSIISYDEKHGVSTTGGRSEAEVMSIYEAWLVKHGKAQSQNSLVEKDRRFEIFKDNLRFVDEHNEKNLSYRLGLTRFADLTNDEYRSKYLGAKMEKKGERRTSLRYEARVGDELPESIDWRKKGAVAEVKDQGGCGSCWAFSTIGAVEGINQIVTGDLITLSEQELVDCDTSYNEGCNGGLMDYAFEFIIKNGGIDTDKDYPYKGVDGTCDQIRKNAKVVTIDSYEDVPTYSEESLKKAVAHQPISIAIEAGGRAFQLYDSGIFDGSCGTQLDHGVVAVGYGTENGKDYWIVRNSWGKSWGESGYLRMARNIASSSGKCGIAIEPSYPIKNGENPPNPGPSPPSPIKPPTQCDSYYTCPESNTCCCLFEYGKYCFAWGCCPLEAATCCDDNYSCCPHEYPVCDLDQGTCLLSKNSPFSVKALKRKPATPFWSQGRKNIA

SEQ ID NO: 32 - protein, 720 aa, Arabidopsis thaliana
  misc_feature (1)..(720): AT2G27100
MADVNLPPSDSVDNRLPEKSTSSSPPPPPPSSSLPQQEQEQDQQQLPLRRERDSRERRDERDIERPPPNRRERDRSPLPPPRRDYKRRPSLSPPPPYRDRRHSPPQRRSPPQKRYRRDDNGYDGRRGSPRGGYGPPDRRFGYDHGGGYDREMGGRPGYGDERPHGRFMGRYQDWEGGRGGYGDASNSGNPQRDGLMSYKQFIQELEDDILPSEAERRYQEYKSEYITTQKRAFFNTHKEEDWLKNKYHPTNLLSVIERRNDLAQKVAKDFLLDLQSGTLDLGPAVTALNKSGRTSEPNSEDEAAGVGKRKRHGMGGAKENELLSAAPKAPSFTSDPKRILTDVEQTQALVRKLDSEKKIEENVLQGSETEKSGREKLHSGSTGPVVIIRGLTSVKGLEGVELLDTLVTYLWRVHGLDYYGKVETNEAKGLRHVRAEGKVSDAKGDENESKFDSHWQERLKGQDPLEVMAAKEKIDAAATEALDPHVRKIRDEKYGWKYGCGAKGCTKLFHAAEFVYKHLKLKHTELVTELTTKVREELYFQNYMNDPNAPGGQPATQQSGPRDRPIRRKPSMENRLRDDRGGRRERDGRANGNDRNDRSEDQQRGDNDGGNPGEVGYDAFGGQGGVHVPPFLSDINPPPMLMPVPGAGPLGPFVPAPPEVAMQMFRDPSGPNPPFEGSGRGGPAPFLLSPAFRQDPRRLRSYQDLDAPEEEVTVIDYRSL

SEQ ID NO: 33 - protein, 916 aa, Arabidopsis thaliana
  misc_feature (1)..(916): AT5G55040
MSQIARRRRKGRPSKADLAARRSSSSPRESGYELRRGQRQRNVRYNFDFDDYFDEEEEDEVEEEKKRQKKLKQVLKLNQSRARADPPVKSRARVRHASDYEEEDEEDDEAEEEEEEVSEKRQVKKRKLNRQDEEEEEEEEKDYDVEEEEEEEEGHADSEEEDDKERKRRSASGNQCDHSSETTPILDKKSLELILDKLQKKDIYGVYAEPVDPEELPDYHDMIEHPMDFSTVRKKLANGSYSTLEELESDVLLICSNAMQYNSSDTVYYKQARTIQEMGKRKFEKARLKIKRAEKELKTDEKVKPDSSVKKQVRQPFSRNGLEAVGSDFSSGANLASGGASQNEPVSTQIGGHEKHSYTDVLFEGNTSLVDSLEKAEDLSSGKGLFGKCGRKLSVVEEDRRATYEDSDQQGDRSESIFTTFESEIKQFVAVGLHAEHAYGRSLARFAATLGPVAWKIASQRIEQALPADFKFGRGWVGEYEPLPTPVLLFETCTPKEPPKKASVFSKRKSNAATKTNETLFKTPLPAKEQQGSRPVRDGNHAFPFPASIGALSEGSPSFVATQVGNLKSMSQHEYRNPSQLDFVKPQNRIPQQVELNLPPPAEQTNSGSGCVLENQSFGKSDTVASYRSSSDMMRNMSSTDSEHYKHQMTTNGIFPGGLRNGKVSPGVNNRMFDLSTDFANQMSRTATSSQQPMRQQSQSHEEQAQIMRNFNERARTQHNSTYNHPKADAPPKISSPQSARSEDSGNASVAAARAWMSIGAGGNNKQTFENASNPKSSQISAESLYNPSREHFHQQAFKPRDAEETQFHPQRNGFPFQTFVHQPVHGMMNGGYQPFQNNRPIVFPQMAAPTSDFSRFHVQSQWRGGITPQVQLKQRQENFNLPPDLNIGVHSPDSPAKQSSGVRVDSQQPDLALQL

SEQ ID NO: 34 - protein, 354 aa, Arabidopsis thaliana
  misc_feature (1)..(354): AT3G03460
MEESKQQQLQQQQLLLQMQQQQQMQQRQQQLFLMQHLQKQQQQQAAMSMFPPNADAHLRPPGLIPNRPVNPFLQNVNPNPNLIQQANKFQQQQQQQMMMMMQQQQLQQQQQQKLMRPSNQLEIQFAYQDAWRVCHPDFKRPFASLEDACERLLPYHVVADYEAEEDDSIFDSNTTSQTLPRCQQWDNNIAAKVAEFTATFEGQVQAFDRIIQKRSDGDRVEERLMMEQVLLNDERNACIQLDREMKAQDARLRMAALAQAAGQARAEESQQSHAEMMARNPLRANAIGNHGEQGRNMNPNEMMLLMNGWGNNNNNNNSQKEEKEPLEDFLNDEENENGEHEKWRRSGDFDLNIR

SEQ ID NO: 35 - protein, 262 aa, Arabidopsis thaliana
  misc_feature (1)..(262): AT1G54390
MAIARTGVYVDDYLEYASTFPAELQRLLNTVRELDERSQSLINQTRQQTKYCLGLASQSSKKGNGNHYNNGGLDEEETIEKMRKEIESSQENALSLCTEKVLLARQAYDLIDSHVKRLDEDLNNFAEDLKQEGKIPPDEPSVLPPLPIVPKAEKRKSFYGTPQPKKIDYRDRDWDRDRDFELMPPPGSNRKDLMPIEEQPIDPNEPTYCVCHQVSFGDMIACDNENCQGGEWFHYTCVGLTPETRFKGKWYCPTCRLLPQSH

SEQ ID NO: 36 - protein, 545 aa, Arabidopsis thaliana
  misc_feature (1)..(545): AT3G20050
MSISAQNPDISGDRQSGQDVRTQNVMACQAVSNIVKTSLGPVGLDKMLVDDIGDVTITNDGATILRMLEVEHPAAKVLVELAELQDREVGDGTTSVVIVAAELLKRANDLVRNKIHPTSIISGYRLAMRESCKYIEEKLVTKVEKLGKVPLINCAKTSMSSKLISGDSDFFANLVVEAVLSVKMTNQRGEIKYPIKGINILKAHGQSARDSYLLNGYALNTGRAAQGMPLRVSPAKIACLDFNLQKTKMQLGVQVVVNDPRELEKIRQREADMTKERIEKLLKAGANVILTTKGIDDMALKYFVEAGAIAVRRVRKEDMRHVAKATGATLVTTFADMEGEETFDPAHLGSADEVVEERIADDDVILIKGTKTSSAVSLILRGANDYMLDEMERALHDALCIVKRTLESNTVVAGGGAVESALSVYLEHLATTLGSREQLAIAEFADALLIIPKVLAVNAAKDATELVAKLRAYHHTAQTKADKKHYSSMGLDLVNGTIRNNLEAGVIEPAMSKVKIIQFATEAAITILRIDDMIKLVKDESQGEE

SEQ ID NO: 37 - protein, 238 aa, Arabidopsis thaliana
  misc_feature (1)..(238): AT4G22320
MEGVGARLGRSSTRYGPATVFTGPVRKWKKKWVHVSPSSKKDNNNSSSGSAAAAASVVNGGSNSDGSNGSHLLLYKWAPLSQNGNGNEDGKSESNSPSEDTVATVAEDPPRRRFKYVPIAVLEEQKKEITEIEEDDKIEEDDKIDEDNKVEQEDKVDEDKTVEESSEKKAEVEVEEKPDINDVPMEDIQQVEEKIVQDDEEKVVRQDLNESTVDLGLNLNANDADAENDPKEDKPLEE

SEQ ID NO: 38 - protein, 535 aa, Arabidopsis thaliana
  misc_feature (1)..(535): AT3G02530
MSVRVLNPNAEVLNKSAALHMTINAAKGLQDVLKSNLGPKGTIKMLVGGSGDIKLTKDGNTLLKEMQIQNPTAIMIARTAVAQDDISGDGTTSTVIFIGELMKQSERCIDEGMHPRVLVDGFEIAKRATLQFLDTFKTPVVMGDEPDKEILKMVARTTLRTKLYEGLADQLTDIVVNSVLCIRKPQEPIDLFMVEIMHMRHKFDVDTRLVEGLVLDHGSRHPDMKRRAENCHILTCNVSLEYEKSEINAGFFYSNAEQREAMVTAERRSVDERVQKIIELKNKVCAGNDNSFVILNQKGIDPPSLDLLAREGIIALRRAKRRNMERLVLACGGEAVNSVDDLTPDCLGWAGLVYEHVLGEEKYTFVEQVKNPHSCTILIKGPNDHTIAQIKDAVRDGLRSVKNTLEDECVVLGAGAFEVAARQHLINEVKKTVQGRAQLGVEAFANALLVVPKTLAENAGLDTQDVIISLTSEHDKGNIVGLDLQDGEPVDPQLAGIFDNYSVKRQLINSGPVIASQLLLVDEVIRAGRNMRKPT

SEQ ID NO: 39 - protein, 536 aa, Arabidopsis thaliana
  misc_feature (1)..(536): AT3G18190
MAAVAAPMASKPRGSKAESFVDNKRREDIRFANINSARAVSDAVRTSLGPKGMDKMISTANGEVIITNDGATILNKMEVLQPAAKMLVELSKSQDSAAGDGTTTVVVIAGALLKECQSLLTNGIHPTVISDSLHKACGKAIDILTAMAVPVELTDRDSLVKSASTSLNSKVVSQYSTLLAPLAVDAVLSVIDPEKPEIVDLRDIKIVKKLGGTVDDTHTVKGLVFDKKVSRAAGGPTRVENAKIAVIQFQISPPKTDIEQSIVVSDYTQMDRILKEERNYILGMIKKIKATGCNVLLIQKSILRDAVTDLSLHYLAKAKIMVIKDVERDEIEFVTKTLNCLPIANIEHFRAEKLGHADLVEEASLGDGKILKITGIKDMGRTTSVLVRGSNQLVLDEAERSLHDALCVVRCLVSKRFLIAGGGAPEIELSRQLGAWAKVLHGMEGYCVKSFAEALEVIPYTLAENAGLNPIAIVTELRNKHAQGEINAGINVRKGQITNILEENVVQPLLVSTSAITLATECVRMILKIDDIVTVR

SEQ ID NO: 40 - protein, 549 aa, Arabidopsis thaliana
  misc_feature (1)..(549): AT3G03960
MVGMSMQPYGIQSMLKEGYRHLSGLDEAVIKNIEACKELSTITRTSLGPNGMNKMVINHLDKLFVTNDAATIVNELEIQHPAAKLLVLAAKAQQEEIGDGANLTISFAGELLQNAEELIRMGLHPSEIISGYTKAVSKAVEILEQLVETGSETMDVRNKDEVISRMRAAVASKQFGQEEIICSLVTDACIQVCPKNPTNFNVDNVRVSKLLGGGLHNSCIVRGMVLKSDAVGSIKRMEKAKVAVFAGGVDTTATETKGTVLIHSAEQLENYAKTEEAKVEELIKAVAESGAKVIVSGGSIGEMALHFCERYKIMVLKISSKFELRRFCRTAGAVAHLKLSRPSPEDLGYVDSISVEEIGGVTVTIARNEEGGNSISTVVLRGSTDSILDDLERAVDDGVNTYKAMCRDSRIVPGAAATEIELAQRLKEYANAEIGLDKYAITKYAESFEFVPKTLADNAGLNAMEIIAALYTGHGSGNTKLGIDLEEGACKDVSETKVWDLFATKLFALKYASDAACTVLRVDQIIMAKPAGGPRRDAAQAAGAGAEED

SEQ ID NO: 41 - protein, 381 aa, Arabidopsis thaliana
  misc_feature (1)..(381): AT3G08580
MVDQVQHPTIAQKAAGQFMRSSVSKDVQVGYQRPSMYQRHATYGNYSNAAFQFPPTSRMLATTASPVFVQTPGEKGFTNFALDFLMGGVSAAVSKTAAAPIERVKLLIQNQDEMIKAGRLSEPYKGIGDCFGRTIKDEGFGSLWRGNTANVIRYFPTQALNFAFKDYFKRLFNFKKDRDGYWKWFAGNLASGGAAGASSLLFVYSLDYARTRLANDAKAAKKGGGGRQFDGLVDVYRKTLKTDGIAGLYRGFNISCVGIIVYRGLYFGLYDSVKPVLLTGDLQDSFFASFALGWVITNGAGLASYPIDTVRRRMMMTSGEAVKYKSSLDAFKQILKNEGAKSLFKGAGANILRAVAGAGVLSGYDKLQLIVFGKKYGSGGA

SEQ ID NO: 42 - protein, 322 aa, Arabidopsis thaliana
  misc_feature (1)..(322): AT4G14880
MASRIAKDVTELIGNTPLVYLNNVAEGCVGRVAAKLEMMEPCSSVKDRIGFSMISDAEKKGLIKPGESVLIEPTSGNTGVGLAFTAAAKGYKLIITMPASMSTERRIILLAFGVELVLTDPAKGMKGAIAKAEEILAKTPNGYMLQQFENPANPKIHYETTGPEIWKGTGGKIDGFVSGIGTGGTITGAGKYLKEQNANVKLYGVEPVESAILSGGKPGPHKIQGIGAGFIPSVLNVDLIDEVVQVSSDESIDMARQLALKEGLLVGISSGAAAAAAIKLAQRPENAGKLFVAIFPSFGERYLSTVLFDATRKEAEAMTFEA

SEQ ID NO: 43 - protein, 103 aa, Arabidopsis thaliana
  misc_feature (1)..(103): AT1G07820
MSGRGKGGKGLGKGGAKRHRKVLRDNIQGITKPAIRRLARRGGVKRISGLIYEETRGVLKIFLENVIRDAVTYTEHARRKTVTAMDVVYALKRQGRTLYGFGG

SEQ ID NO: 44 - protein, 512 aa, Arabidopsis thaliana
  misc_feature (1)..(512): AT2G47620
MEATDPSAEIELYTIPAQSSWFLWDDIHEIERREFAEFFTESSITRTPKVYKEYRDFIINKFREDTCRRLTFTSVRKFLVGDVNLLQKVFLFLEKWGLINFSSSLKKNDHLLSVDNAKIEQGTPAGIRVTATPNSLRPITAPPLVEERVETGIKVPPLTSYSDVFSDLKKPDHVLVCAHCGERCDSPFYQHNKGIVNICEKCFKNGNYGENNTADDFKLIGNSAAAVWTEEEILLLLESVLKHGDDWELISQSVSTKSRLDCISKLIELPFGEFLMGSASGRLNPSILTEDENTEQVQTDGQEHEETETREEKEDRVNEDEPPAKRKRVALISEGDSSLMKQVAAMASKVGPSVATAAAKAALAALCDEASCPKEIFDTDDYSNFTVDRANGEKDTDMEEQQEEKDGPQGLPVALRIRASVATALGAAAAQAKILADQEEREMEQLAATVIEQQLKKLQSKLKFLDDLESIMDEEEKVIEGVKETIIQERVSVLQCAFRSGITKRWDHTYVK

SEQ ID NO: 45 - protein, 469 aa, Arabidopsis thaliana
  misc_feature (1)..(469): AT2G33610
MAMKAPDPGGSGEILPSTPSLSETTSGGAAAASKSAQLPSSSSDIDNIHVPSYSSWFSWTDINDCEVRSLPEFFDSRSSSKNPKFYLYLRNSIIKQYRDDHPRKISFTDVRRTLVSDVVSIRRVFDFLDSWGLINYNSSASAKPLKWEEKEAGKSAGDAASEPATTVKETAKRNCNGCKAICSIACFACDKYDLTLCARCYVRSNYRVGINSSEFKRVEISEESKPEWSDKEILLLLEAVMHYGDDWKKVASHVIGRTEKDCVSQFVKLPFGEQFVKESDSEDGLEMFDQIKDSDIPESEGIDKDGSSPNKRIKLTPLADASNPIMAQAAFLSALAGTNVAEAAARAAVRALSDVDYEADKNASRDPNRQDANAASSGETTRNESERAWADAKSLIEKEEHEVEGAIKETVEVEMKKIRDRIVHFEKLDLEMERSRKQLEEVRNLLFVDQLNIFFHTRKARKTEDRIEC

SEQ ID NO: 46 - protein, 240 aa, Arabidopsis thaliana
  misc_feature (1)..(240): AT3G17590
MKGLVSTGWKGPVKFRMPTAENLVPIRLDIQFEGQRYKDAFTWNPSDPDNEVVIFAKRTVKDLKLPYAFVTQIAQSIQSQLSDFRAYEGQDMYTGEKIIPIKLDLRVNHTLIKDQFLWDLNNFESDPEEFARTLCKDLGVEDPEVGPAVAFAIREQLYEIAIQSVASARESRLSKKGRRGSDHGSASKASGLSMDLMKLFSFKSSVVRKRKDLDVYEPVVDLLTSEEVDALEAREERHAR

SEQ ID NO: 47 - protein, 985 aa, Arabidopsis thaliana
  misc_feature (1)..(985): AT4G34430
MEEKRRDSAGTLAFAGSSGDSPASEPMPAPRRRGGGLKRKANALGGSNFFSSAPSKRMLTREKAMLASFSPVHNGPLTRARQAPSIMPSAADGVKSEVLNVAVGADGEKPKEEEERNKAIREWEALEAKIEADFEAIRSRDSNVHVVPNHCGWFSWEKIHPLEERSLPSFFNGKLEGRTSEVYREIRNWIMGKFHSNPNIQIELKDLTELEVGDSEAKQEVMEFLDYWGLINFHPFPPTDTGSTASDHDDLGDKESLLNSLYRFQVDEACPPLVHKPRFTAQATPSGLFPDPMAADELLKQEGPAVEYHCNSCSADCSRKRYHCPKQADFDLCTECFNSGKFSSDMSSSDFILMEPAEAPGVGSGKWTDQETLLLLEALEIFKENWNEIAEHVATKTKAQCMLHFLQMPIEDAFLDQIDYKDPISKDTTDLAVSKDDNSVLKDAPEEAENKKRVDEDETMKEVPEPEDGNEEKVSQESSKPGDASEETNEMEAEQKTPKLETAIEERCKDEADENIALKALTEAFEDVGHSSTPEASFSFADLGNPVMGLAAFLVRLAGSDVATASARASIKSLHSNSGMLLATRHCYILEDPPDNKKDPTKSKSCSADAEGNDDNSHKDDQPEEKSKKAEEVSLNSDDREMPDTDTGKETQDSVSEEKQPGSRTENSTTKLDAVQEKRSSKPVTTDNSEKPVDIICPSQDKCSGKELQEPLKDGNKLSSENKDASQSTVSQSAADASQPEASRDVEMKDTLQSEKDPEDVVKTVGEKVQLAKEEGANDVLSTPDKSVSQQPIGSASAPENGTAGGNPNIEGKKEKDICEGTKDKYNIEKLKRAAISAISAAAVKAKNLAKQEEDQIRQLSGSLIEKQLHKLEAKLSIFNEAESLTMRVREQLERSRQRLYHERAQIIAARLGVPPSMSSKASLPTNRIAANFANVAQRPPMGMAFPRPPMPRPPGFPVPGSFVAATTMTGSSDPSPGSDNVSSV

SEQ ID NO: 48 - protein, 327 aa, Arabidopsis thaliana
  misc_feature (1)..(327): AT1G32730
MATSSPSLSNNGLSSVVTPPKTLRGLNKPKCIQCGNVARSRCPFQSCKGCCSRAENPCPIHVLKVASTSGEKTQAPSTPSSEQKATEGTPGSTTRVSSIRQLSSNFAQFNNLNASSRQRKPLTIKDAQALNEWRFTKLKEYRDRNIEVENEAFDRYMSNVNLLEEAFSFTSVPDEESHGTAAPEQNKEENIVSELKLRLRSNSARTESFKKRIAETVKAGLVKLKRLDLGSSSDDQDDIKRRVKRKKWEEKGSALNEIIDKLNKARTEEDLKSCLEMKSKLCGQVSPTAASEKNKIFPGVVRKVEMSEEALQKIAENLQSFDKVGML

SEQ ID NO: 49 - protein, 136 aa, Arabidopsis thaliana
  misc_feature (1)..(136): AT1G06500
MMHSTVQHGGNKSGKSNVWANTNLAKTVAAVDEFKFGFPSGGLTTVSNKWWGRAEKGGREDGGGENTENGHVAACDETQNSLVAIRKRIAEEGREAVELGLHQGFGSKRPGKRDQALLFQIFNSAMPKDWVTPDSS

SEQ ID NO: 50 - protein, 460 aa, Arabidopsis thaliana
  misc_feature (1)..(460): AT3G22990
MQKRELGKSGGNSGGSSGPPAKRGRPFGSTSANSAAAAAAAAAADAMSPSALLGPSLLVHNSFVEQNNRRIVLALQSGLKSEVTWALNTLTLLSFKEKEDIRRDVMPLAKIAGLLDALLLIIDDWRDIALPKDLTRGTRVRTLGTNASVTGFGNEYDALASIQPPGSGIGSSAAEALGKKSTGKHQSSQWWMEEDGLFNLDDEGRSEKQMCAIAASNVIRNFSFMPDNEVVMAQHRHCLETVFQCIHDHMTEDEELVTNSLETIVNLAHLMDLRIFSSLKQSYININEKKAVQAVVGILNSSVKAWNCAAAELLGRLIINPDNEPFISPLIPQIHKRLIDLLSIQAVDAQAAAVGALYNLVEVNMDCRLKLASERWAVDRLLKVIKTPHPVPEVCRKAAMILENLVSEPQNRGLLLAYENAFAELLFQEGKYSDSFARILYELTARSNSRVASARGIWGM

SEQ ID NO: 51 - protein, 348 aa, Arabidopsis thaliana
  misc_feature (1)..(348): AT3G18380
MGRPPSNGGPAFRFILPEVTEMEAILLQHNTAMPGRHILEALADKFSESPERKGKVVVQFKQIWNWFQNRRYALRARGNKAPGKLNVSSMPRMDLPNQMRSVIQPLSVPKTTHMTGNLPGMTPAPSGSLVPGVMRSGSDNSYLEFEAKSARDGAWYDVQAFLAHRNLEIGDPEVQVRFAGFEVEEDEWINVKKHVRQRSLPCEASECVAVLAGDLVLCFQEGKDQALYFDAIVLDAQRRRHDVRGCRCRFLVRYSHDQSEEIVPLRKICRRPETDYRLQQLHNAVNDLANSNQHQIPALDAAAKTPLSLPGATVPIVAPESKDPSLSATPATLVQPSSNAATVPAGSA

SEQ ID NO: 52 - protein, 1132 aa, Arabidopsis thaliana
  misc_feature (1)..(1132): AT3G06010
MVAQQLQERCGGTSQEDPVETTKSLICALNYISRDLPLPPHLFTAVSSIYHGASSSSLSDSDVSPPLPTSPPANKAPYGADLMGEFEDALLKQRPDCESGSRLIQLLDNRNKSHIQRRLSELEGGCFVLTLCVFESELPSTRGEDLQAKCLLELYGLKLRELQGKVRTAVSSEFWLRLNCADVSSQVFDWGMMRLPRPFYGVGDPFAMEADDQFRKKRDAELSIFVIGIADVLKVFVQRLSRLEEEEKNLIETAKRKFFAEVLNAVREFQLQIQATQKRRRQRNDGVQAWHGRQRQRATRAEKLRLMALKSDDQEAYMKLVKESKNERLTTLLEETNKLLANLGAAVQRQKDAKLPEGIDLLKDSESDLSELDAPRSEPLQDLLPDQDIDITESDNNDDSNDLLEGQRQYNSAIHSIQEKVTEQPSLLEGGELRSYQLEGLQWMVSLFNNNLNGILADEMGLGKTIQTISLIAYLLENKGVPGPYLIVAPKAVLPNWVNEFATWVPSIAAFLYDGRLEERKAIREKIAGEGKFNVLITHYDLIMRDKAFLKKIEWYYMIVDEGHRLKNHESALAKTLLTGYRIKRRLLLTGTPIQNSLQELWSLLNFLLPHIFNSVQNFEEWFNAPFADRGNVSLTDEEELLIIHRLHHVIRPFILRRKKDEVEKFLPGKTQVILKCDMSAWQKVYYKQVTDMGRVGLQTGSGKSKSLQNLTMQLRKCCNHPYLFVGGDYNMWKKPEIVRASGKFELLDRLLPKLRKAGHRILLFSQMTRLIDVLEIYLTLNDYKYLRLDGTTKTDQRGLLLKQFNEPDSPYFMFLLSTRAGGLGLNLQTADTVIIFDSDWNPQMDQQAEDRAHRIGQKKEVRVFVLVSVGSVEEVILERAKQKMGIDAKVIQAGLFNTTSTAQDRREMLEEIMRKGTSSLGTDVPSEREINRLAARSEDEFWMFERMDEERRRKENYRARLMQEQEVPEWAYTTQTQEEKLNNGKFHFGSVTGKRKRKEIVYSDTLSELQWLKAVESGEDLSKLSMRYNRREENASNTKTSTSKKVIESIQTVSDGTSEEDEEEQEEERAKEMSGKQRVDKSEEEEEEGEEENDGKAIFKWNTHKKKRSRYSFTCSSSDSRAQSSNGSRRK

SEQ ID NO: 53 - protein, 572 aa, Arabidopsis thaliana
  misc_feature (1)..(572): AT1G58025
MKRKRGHKKGKKSKTINEQGNLNESTENAENQTSEHSSEAPVECENSELESKMEVDAPSPIAGSVDLADNIAAKSVARVKVKLKTSKAPEPNETLRDDIDKSSSQAVLEKPVVPVEKKEEFVPRLPERKPVFLNVYRKTKGIRIKSSKAVDGSSSVTEKSATDTVKVQDVVVGQKDTKTSEENSQASKKEAEIATISLQKEEKKTDQNLRYNKQELEDSLIVIKKIMKMEAADPFNVPVNPEALGIPDYFDIIKTPMDFGTICNNFEKGNKYMNSEDVYKDVNYIWNNCSKYNKKGDYIVDLMKRVKKNFMKYWTSAGLYTEQSAENTEDGGKASTKQKSHKRHGRHHKSDCMCAICVLKRRKRERERDSGAQEESSPAGSPSVDNSSVNMGEDMDIDVDKKPEQEKTEIVELDSPVSKTQRVIENKQEVEEEENVEVESENKTKANVEDKTQSIDRSMEETGDEPVNSAAEKLVVLASLEGPKSTQNEEEEKEKRLQEQKKRLELERKEWRMKMQEKFQVRNPQLLSLCETLFPNDNNHNSVWNGPHSLFRRRGGSNRSSALHKAVESLMK

SEQ ID NO: 54 - protein, 374 aa, Arabidopsis thaliana
  misc_feature (1)..(374): AT5G03290
MTMAANLARRLIGNRSTQILGAVNSSSGAASSVARAFCSSTTPITATLFPGDGIGPEIAESVKKVFTTAGVPIEWEEHYVGTEIDPRTQSFLTWESLESVRRNKVGLKGPMATPIGKGHRSLNLTLRKELNLYANVRPCYSLPGYKTRYDDVDLITIRENTEGEYSGLEHQVVRGVVESLKIITRQASLRVAEYAFLYAKTHGRERVSAIHKANIMQKTDGLFLKCCREVAEKYPEITYEEVVIDNCCMMLVKNPALFDVLVMPNLYGDIISDLCAGLVGGLGLTPSCNIGEDGVALAEAVHGSAPDIAGKNLANPTALLLSGVMMLRHLKFNEQAEQIHSAIINTIAEGKYRTADLGGSSTTTEFTKAICDHL

SEQ ID NO: 55 - protein, 403 aa, Arabidopsis thaliana
  misc_feature (1)..(403): AT3G50000
MHLIFFFSYFLRRYLLLLCAILILRAPLAHSLIPPLTCVNTGTVESDVTGIRFDRCLDTDSLAKISLSTVMSKARVYTDVNVIRPKDYWDYESLNVQWGEQDDYEVVRKVGRGKYSEVFEGINMNNNEKCIIKILKPVKKKKIRREIKILQNLCGGPNIVKLLDVVRDQHSKTPSLIFEYVNSTDFKVLYPTLTDYDIRYYIYELLKALDFCHSQGIMHRDVKPHNVMIDHELRKLRLIDWGLAEFYHPGKEYNVRVASRYFKGPELLVDLQDYDYSLDMWSLGCMFAGMIFRKEPFFYGHDNQDQLVKIAKVLGTDELNAYLNKYQLELDTQLEALVGRHSRKPWSKFINADNRHLVSPEAIDYLDKLLRYDHQDRLTAKEAMAHPYFAQVRAAESSRMRTQ

SEQ ID NO: 56 - protein, 283 aa, Arabidopsis thaliana
  misc_feature (1)..(283): AT1G11840
MAEASDLLEWPKKDNRRFLHVVYRVGDLDRTIEFYTEVFGMKLLRKRDIPEEKYSNAFLGFGPETSNFVVELTYNYGVSSYDIGTGFGHFAISTQDVSKLVENVRAKGGNVTREPGPVKGGGSVIAFVKDPDGYTFELIQRGPTPEPFCQVMLRVGDLDRAIKFYEKALGMRLLRKIERPEYKYTIGMMGYAEEYESIVLELTYNYDVTEYTKGNAYAQIAIGTDDVYKSGEVIKIVNQELGGKITREAGPLPGLGTKIVSFLDPDGWKTVLVDNKDFLKELE

SEQ ID NO: 57 - protein, 520 aa, Arabidopsis thaliana
  misc_feature (1)..(520): AT4G04740
MGCFSSKHRKTQNDGGGERSIPIIPVQTHIVDQVPDHRKPQIPSPSIPISVRDPETILGKPFEDIRKFYSLGRELGRGGLGITYMCKEIGTGNIYACKSILKRKLISELGREDVKTEIQIMQHLSGQPNVVEIKGSYEDRHSVHLVMELCAGGELFDRIIAQGHYSERAAAGTIKSIVDVVQICHLNGVIHRDLKPENFLFSSKEENAMLKVTDFGLSAFIEEGKIYKDVVGSPYYVAPEVLRQSYGKEIDIWSAGVILYILLCGVPPFWADNEEGVFVEILKCKIDFVREPWPSISDSAKDLVEKMLTEDPKRRITAAQVLEHPWIKGGEAPEKPIDSTVLSRMKQFRAMNKLKKLALKVSAVSLSEEEIKGLKTLFANMDTNRSGTITYEQLQTGLSRLRSRLSETEVQQLVEASDVDGNGTIDYYEFISATMHRYKLHHDEHVHKAFQHLDKDKNGHITRDELESAMKEYGMGDEASIKEVISEVDTDNDGKINFEEFRAMMRCGTTQPKGKQYPFH

SEQ ID NO: 58 - protein, 524 aa, Arabidopsis thaliana
  misc_feature (1)..(524): AT4G28520
MVKLSNLLVATFGVLLVLNGCLARQSLGVPPQLQNECNLDNLDVLQATETIKSEAGQIEYWDHNHPQLRCVGVSVARYVIEQGGLYLPTFFTSPKISYVVQGTGISGRVVPGCAETFMDSQPMQGQQQGQPWQGRQGQQGQPWEGQGQQGQQGRQGQPWEGQGQQGQQGRQGQQGQPWEGQGQQGQQGFRDMHQKVEHVRRGDVFANTPGSAHWIYNSGEQPLVIIALLDIANYQNQLDRNPRVFHLAGNNQQGGFGGSQQQQEQKNLWSGFDAQVIAQALKIDVQLAQQLQNQQDSRGNIVRVKGPFQVVRPPLRQPYESEEWRHPRSPQGNGLEETICSMRSHENIDDPARADVYKPSLGRVTSVNSYTLPILEYVRLSATRGVLQGNAMVLPKYNMNANEILYCTGGQGRIQVVNDNGQNVLDQQVQKGQLVVIPQGFAYVVQSHGNKFEWISFKTNENAMISTLAGRTSLLRALPLEVISNGFQISPEEARKIKFNTLETTLTRAAGRQQQQLIEEIVEA

SEQ ID NO: 59 - protein, 472 aa, Arabidopsis thaliana
  misc_feature (1)..(472): AT5G44120
MARVSSLLSFCLTLLILFHGYAAQQGQQGQQFPNECQLDQLNALEPSHVLKSEAGRIEVWDHHAPQLRCSGVSFARYIIESKGLYLPSFFNTAKLSFVAKGRGLMGKVIPGCAETFQDSSEFQPRFEGQGQSQRFRDMHQKVEHIRSGDTIATTPGVAQWFYNDGQEPLVIVSVFDLASHQNQLDRNPRPFYLAGNNPQGQVWLQGREQQPQKNIFNGFGPEVIAQALKIDLQTAQQLQNQDDNRGNIVRVQGPFGVIRPPLRGQRPQEEEEEEGRHGRHGNGLEETICSARCTDNLDDPSRADVYKPQLGYISTLNSYDLPILRFIRLSALRGSIRQNAMVLPQWNANANAILYVTDGEAQIQIVNDNGNRVFDGQVSQGQLIAVPQGFSVVKRATSNRFQWVEFKTNANAQINTLAGRTSVLRGLPLEVITNGFQISPEEARRVKFNTLETTLTHSSGPASYGRPRVAAA